(19) United States
(12) Patent Application Publication
(10) Pub. No.: US 2024/0287232 A1
(43) Pub. Date: Aug. 29, 2024

(54) BIOSTABLE POLYMER BRUSHES WITH DEFINED VISCOSITY AND OPTICAL PROPERTIES FOR USE IN A NOVEL INTRAOCULAR LENS

(71) Applicants: DUKE UNIVERSITY, Durham, NC (US); ADAPTILENS, LLC, Chestnut Hill, MA (US)

(72) Inventors: Matthew L. Becker, Chapel Hill, NC (US); Metin KARAYILAN, Durham, NC (US); Liane CLAMEN, Chestnut Hill, MA (US)

(21) Appl. No.: 18/562,590

(22) PCT Filed: May 20, 2022
(86) PCT No.: PCT/US2022/030255

§ 371 Date: Nov. 20, 2023

Related U.S. Application Data
(60) Provisional application No. 63/191,018, filed on May 20, 2021.

(51) Int. Cl. C08F 230/08; A61F 2/16; A61F 9/00; A61K 9/00

(52) U.S. Cl. CPC C08F 230/085; A61F 2/16; A61F 9/0017; A61K 9/0051; C08F 2438/00

(57) ABSTRACT

In one or more embodiments, the present invention provides a bottlebrush polymer for use with implantable synthetic intraocular lenses comprising a homopolymer or copolymer of a high reflective index methacrylate monomer or macromolecule such as monomethacryloxypropyl terminated polydimethylsiloxane, asymmetric (PDMS-MA), 2,2,2-trifluoroethyl methacrylate (TFEMA), oligo(ethylene glycol) methacrylate (OEGMA), 3,3,4,4,5,5,6,6,7,7,8,8,9,9,10,10,10-heptadecafluorodecyl methacrylate (HDFDMA), benzyl methacrylate (BzMA), 2-[3-(2H-Benzotriazol-2-yl)-4-hydroxyphenyl]ethyl methacrylate (BzTAzMA), ethyleneglycol phenylether methacrylate (EGPhEMA), hydroxyethyl methacrylate (HEMA), or a combinations thereof having a refractive index and viscosity suitable for use as a filling material for implantable synthetic intraocular lenses.

### CROSS REFERENCE TO RELATED APPLICATIONS

This application claims the benefit of U.S. provisional patent application Ser. No. 63/191.018 entitled “Biostable Polymer Brushes with Defined Viscosity and Optical Properties for Use in a Novel Intraocular Lens,” filed May 20, 2021, and incorporated herein by reference in its entirety.

### NAMES OF THE PARTIES TO A JOINT RESEARCH AGREEMENT

The present application stems from work done pursuant to a Joint Research Agreement between Duke University of Durham, N.C. and Adaptilens, LLC of Chestnut Hill. Massachusetts.

### FIELD OF THE INVENTION

One or more embodiments of the present invention relate to bottlebrush polymers. In certain embodiments, the invention relates to biostable polymer bottlebrushes that have tunable viscosity and optical properties for use in intraocular lenses.

### BACKGROUND OF THE INVENTION

Polymers are split into two general classes: thermoplastics and thermosets. Due to lacking a crosslinked network, thermoplastics can be soluble in good solvents and become soft or melt when heated, in this way they can be reprocessable and remoldable. Thermosets, on the other hand, contain crosslinked networks and are irreversibly cured for high-performance applications. Elastomers are a class of materials that contain lightly crosslinked polymer networks which give elasticity to the elastomers. Soft elastomers can be prepared by increasing the molecular weight of the network strand (polymer chains between two junctions/crosslinking points) and by decreasing the chain entanglement of the polymer chains. Polymer chains start generating entanglement within the system above a certain molecular weight called chain entanglement molecular weight, and these entangled chains are permanently trapped upon crosslinking which then behave as topological crosslinks. These chain entanglements can be prevented or delayed by changing the “volume” of the polymer chain which is possible through changing the architecture of the polymer chain from linear to branched or regime where the side chains are highly stretched and crowded which forces the backbone into a highly extended state, commonly referred to as bottlebrush.

Bottlebrush polymers (BBPs) are a type of polymers with long and densely grafted side chains. BBPs can be synthesized using different approaches such as grafting to, grafting from, and grafting through approaches. In the grafting to approach, long polymer chains asymmetrically terminated with a functional group can be chemically connected to a polymer backbone with many functional groups ideally on every repeating unit which are reactive to the functional group on the long polymer chains. The grafting from approach requires a polymer backbone with initiator sites ideally on every repeating unit. Using small-molecule monomers, polymer side chains can be grown from the polymer backbone, typically using Controlled Radical Polymerization (CRP) techniques such as Atom Transfer Radical Polymerization (ATRP) and Reversible Addition-Fragmentation Chain Transfer (RAFT) Polymerization. Finally, the grafting through approach allows using macromonomers with a polymerizable unit on one chain end and utilizing different polymerization techniques such as reversible deactivation radical polymerization (RDRP; i.e., ATRP or RAFT) or ring-opening metathesis polymerization (ROMP). While RDRP requires commonly used styrenic or (meth)acrylic functional groups, ROMP utilizes norbornene-based polymer chain ends to polymerize macromonomers into BBPs.

The different approaches discussed earlier have given rise to different grafting densities. Generally, the grafting to approach gives the lowest grafting density and the grafting through approach produces the highest grafting density among these three approaches. The difference in the grafting density due to the selection of different grafting approaches stems from the steric hindrance generated between the components (i.e., between polymer backbone-long side chain for grafting to, growing chains on the polymer backbone for grafting from, and growing BBPs-macromonomers for grafting through). To synthesize true BBPs, the grafting density should be high enough that it gives the BBPs rigidity and prevents entanglement. BBPs and bottlebrush gels can be differentiated depending on the macromonomer identity (the side chain plays a central role to determine the final properties of the resulting BBPs), side-chain degree of polymerization (DP), backbone DP, grafting density (distance between each side chain), and crosslinking density (for bottlebrush gels).

Recent developments in the technology of certain intraocular lenses (IOLs) and accommodating or adaptive intraocular lenses (A-IOLs) for use in surgery for treating cataracts have created a need for optically clear, biostable, polymer filling materials having a suitable refractive index (nᵣ) and complex viscosity. Over time, the lens in the eye becomes stiffer and less flexible, making it more difficult for the eye to focus on near objects. This gradual, age-related loss of accommodation is called presbyopia. As people age further, the lens becomes thickened and opaque, forming a cataract and causing blurred vision. The standard of care is to have cataract surgery to remove the cataract and replace it with an IOL. The current standard lenses are flat monofocal IOLs which cannot adjust for both near and far sight, leaving patients dependent upon glasses. A-IOLs would allow patients to see clearly over a range of distances without eyeglasses or contact lenses. Various different polymers have been used in lens refilling IOLs and in other types of A-IOLs with mixed results. Many of these materials require solvents or other diluting fluids to arrive at working viscosities. Both Jean Marie Parel, PhD (Bascom Palmer) and Steven Koopmans, Md. (Pharmacia) attempted to restore accommodation by refilling the capsular bag with a soft polymer (Hao et al., 2010; Koopmans, 2003 and 2006). They both injected in situ polymerizing materials directly into the capsular bag. Both scientists ended their efforts after in vivo animal trials presented significant complications including inflammation of the eye and posterior capsular opacification (PCO) (Koopmans, 2014, Hao 2012). Similarly, Nishi's attempts at developing an endocapsular balloon filled with silicone oil failed when severe posterior capsule opacification occurred (Nishi 1997). The Fluid Vision IOL is a hydrophobic acrylic lens with a hollow optic and two hollow haptics filled with silicone oil. When the ciliary muscles contract, the oil shifts from the haptics into the optic to change the shape of the lens. Although this IOL is still in Phase II clinical trials, problems with the lens include the slow speed at which patients focus and the inconsistent effective lens position (Young, 2016).

What is needed in the art is a synthetic route to generating optically clear, biostable polymer bottlebrushes that have tunable viscosities and optical properties that can be used as a fluid-like filler material in IOLs and A-IOLs.

### SUMMARY OF THE INVENTION

In one or more embodiments, the present invention provides a synthetic route to generating optically clear, biostable bottlebrush polymers that have tunable mechanical and optical properties making them suitable for use as a fluid-like filler material in implantable intraocular lenses for use in treating presbyopia, cataracts, and similar maladies. The method controls with precision the refractive index of the bottlebrush polymers and their mechanical properties necessary for the fluid to fit into the lens, while avoiding the use of solvents or other diluting fluids.

In various embodiments, the present invention uses RAFT polymerization and a grafting through approach to make BBPs for use in intraocular lenses (IOLs). In one or more of these embodiments, two methacrylate macromonomers with low glass transition temperatures (T_{g}), different refractive indices (nᵣ), and hydrophobicity are used. In some of these embodiments, poly(dimethylsiloxane)-methacrylate (PDMS-MA) and/or oligo(ethylene glycol) methacrylate (OEGMA) are used. PDMS-MA is a hydrophobic macromonomer with nᵣ of 1.41-142. Oligo(ethylene glycol) methacrylate (OEGMA), on the other hand, is a hydrophilic macromonomer with nᵣ of 1.45-1.46. In some embodiments, these two macromonomers can be homopolymerized to yield poly(PDMS-MA) and poly(OEGMA) or copolymerized to yield poly(PDMS-MA-random-OEGMA) all of which are honey-like viscous liquids with a complex viscosity in the range of 0.4-12 Pa·s. It has also been found that low and high nᵣ small molecule methacrylate monomers can be copolymerized with PDMS-MA and OEGMA to tune the final nᵣ without increasing the viscosity outside of the range. So, in some embodiments, fluorinated methacrylic monomers are copolymerized with PDMS-MA to decrease the nᵣ and benzylic monomers are copolymerized with OEGMA to reach the upper limit of nᵣ. In these embodiments, the final nᵣ range is 1.40-1.48. Additionally, it has been found that using the similar copolymerization approach, it is possible to incorporate a UV-absorbing reagent into the BBP backbone to filter out UV light.

In one or more embodiments, the optically clear, biostable bottlebrush polymers of the present invention may be used to create an A-IOL having a thin flexible shell and a filling material comprising the optically clear, biostable bottlebrush polymers. When the ciliary muscles of the eye contract during accommodation, the flexible lens will change shape such that the power of the lens will increase and allow the patient to focus at near. Once the muscles of accommodation relax, the lens will resume its baseline shape, allowing the patient to see at distance.

The IOL described herein is advantageous because compared to other devices, it utilizes natural accommodation to vary precisely the optical power of the eye without damaging the tissue thereof, or the circulating aqueous materials. In a preferred embodiment the IOL is soft and flexible to ensure the IOL-eye system re-establishes the accommodative mechanism so that the optical system of the patient can respond to changes in spatial images and illumination; permitting the lens to be installed by a simple procedure that can be quickly performed. In addition, the IOL localizes in the natural capsule so as to minimize de-centering and accommodation loss; providing functional performance similar to a natural eye; and allowing volumetric accommodation so that the ciliary muscle can control accommodation of the IOL. As a result, a greater variety of patients with lens disease can be provided with natural, responsive acuity, under a greater variety of circumstances, including but not limited to, enhanced capacity for accommodation, reduced glare, and permanent functionality because it utilizes a novel system of polymeric shell and filling material to enhance the optical performance of the eye and establish normal visual experiences.

In a first aspect, the present invention is directed to a bottlebrush polymer comprising a homopolymer of a methacrylate macromolecule monomer selected from the group consisting of monomethacryloxypropyl terminated polydimethylsiloxane, asymmetric (PDMS-MA) and oligo(ethylene glycol) methacrylate (OEGMA) or a copolymer of at least one of PDMS-MA and OEGMA and at least one methacrylate or acrylate monomer may include, without limitation, 2,2,2-trifluoroethyl methacrylate (TFEMA), 3,3,4,4,5,5,6,6,7,7,8,8,9,9,10,10,10-heptadecafluorodecyl methacrylate (HDFDMA), benzyl methacrylate (BzMA), 2-[3-(2H-Benzotriazol-2-yl)-4-hydroxyphenyl]ethyl methacrylate (BzTAzMA), ethyleneglycol phenylether methacrylate (EGPhEMA), hydroxyethyl methacrylate (HEMA), 2,2,2-trifluoroethyl acrylate (TFEA), 3,3,4,4,5,5,6,6,7,7,8,8,9,9,10,10,10-heptadecafluorodecyl acrylate (HDFDA), benzyl acrylate (BzA), 2-[3-(2H-Benzotriazol-2-yl)-4-hydroxyphenyl]ethyl acrylate (BzTAZA), ethyleneglycol phenylether acrylate (EGPhEA), hydroxyethyl acrylate (HEA), and combinations thereof, and having one or more end groups derived from a reversible addition fragmentation chain-transfer (RAFT) agent.

In one or more embodiments, the homopolymer or copolymer comprises the residue of an ultra-violet (UV) light blocking methacrylate monomer. In one or more of these embodiments, the ultra-violet light blocking methacrylate monomer is 2-[3-(2H-Benzotriazol-2-yl)-4-hydroxyphenyl]ethyl methacrylate (BzTAzMA).

In one or more embodiments, the bottlebrush polymer of the present invention includes any one or more of the above referenced embodiments of the first aspect of the present invention wherein the bottlebrush polymer is a copolymer of monomethacryloxypropyl terminated polydimethylsiloxane, asymmetric (PDMS-MA) and oligo(ethylene glycol) methacrylate (OEGMA) formed by RAFT polymerization and comprising from about 10 to about 95 mole percent, preferably from about 10 to 90 mole percent, and more preferably from about 10 to about 80 mole percent PDMS-MA.

In one or more embodiments, the bottlebrush polymer of the present invention includes any one or more of the above referenced embodiments of the first aspect of the present invention having a complex viscosity of from about 0.5 to about 30 Pa·s at 37° C. In one or more embodiments, the bottlebrush polymer of the present invention includes any one or more of the above referenced embodiments of the first aspect of the present invention having a refractive index of from about 1.39 to about 1.48, preferably from about 1.40 to about 1.46, and more preferably from about 1.42 to about 1.46 at 37° C.

In one or more embodiments, the bottlebrush polymer of the present invention includes any one or more of the above referenced embodiments of the first aspect of the present invention wherein the RAFT agent is selected from the group consisting of dithiobenzoates, trithiocarbonates, and combinations thereof. In one or more embodiments, the bottlebrush polymer of the present invention includes any one or more of the above referenced embodiments of the first aspect of the present invention wherein the RAFT agent has a formula selected from:

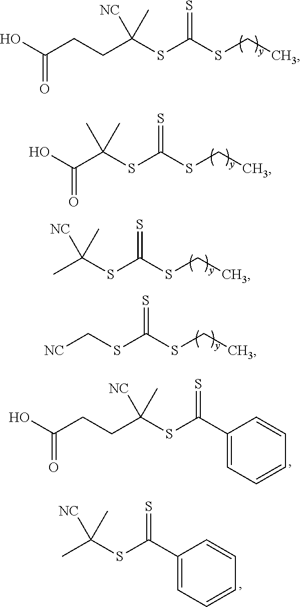

and combinations thereof, where y is an integer from about 3 to about 11.

In one or more embodiments, the bottlebrush polymer of the present invention includes any one or more of the above referenced embodiments of the first aspect of the present invention having the formula:

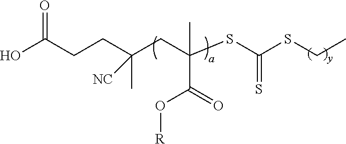

wherein R has the formula

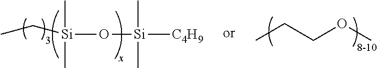

where x is an integer from about 5 to about 10; y is an integer from about 3 to about 11; and a is an integer from about 20 to about 300. In one or more embodiments, the bottlebrush polymer of the present invention includes any one or more of the above referenced embodiments of the first aspect of the present invention having the formula:

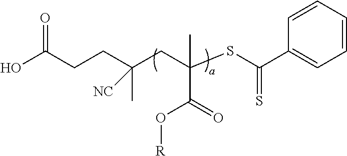

wherein R has the formula

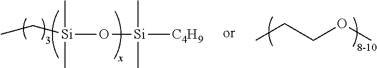

where x is an integer from about 5 to about 10; and a is an integer from about 20 to about 300.

In one or more embodiments, the bottlebrush polymer of the present invention includes any one or more of the above referenced embodiments of the first aspect of the present invention having the formula:

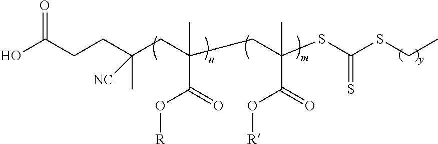

where R has the formula

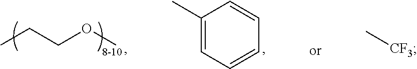

R′ has the formula

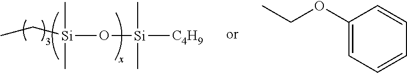

where x is an integer from 5 to 10; y is an integer from about 3 to about 11; n is a mole percent from about 70% to about 95%; and m is a mole percent from about 5% to about 30%. In one or more embodiments, the bottlebrush polymer of the present invention includes any one or more of the above referenced embodiments of the first aspect of the present invention having the formula:

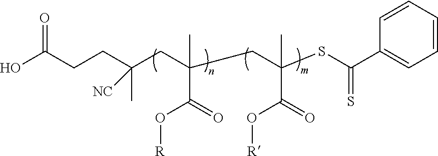

wherein R has the formula

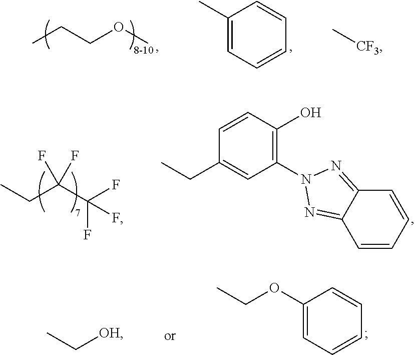

R′ has the formula

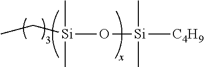

where x is an integer from about 5 to about 10; n is a mole percent from about 5% to about 30%; and m is an mole percent from about 70% to about 95% and n+m=100.

In one or more embodiments, the bottlebrush polymer of the present invention includes any one or more of the above referenced embodiments of the first aspect of the present invention having the formula:

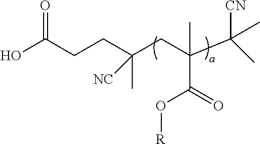

where R has the formula

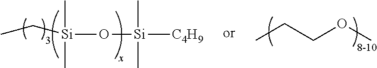

where y is an integer from about 5 to about 10; and a is an integer from about 20 to about 300. In one or more embodiments, the bottlebrush polymer of the present invention includes any one or more of the above referenced embodiments of the first aspect of the present invention having the formula

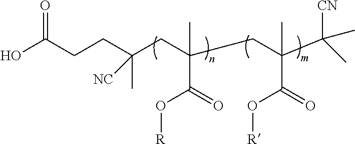

where R has the formula

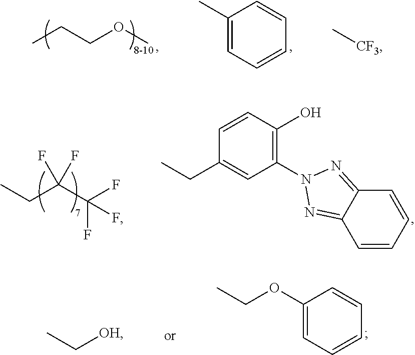

R′ has the formula

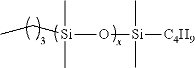

where x is an integer from about 5 to about 10; n is a mole percent from about 70% to about 95%; and m is an mole percent from about 5% to about 30%.

In one or more embodiments, the bottlebrush polymer of the present invention includes any one or more of the above referenced embodiments of the first aspect of the present invention having the formula:

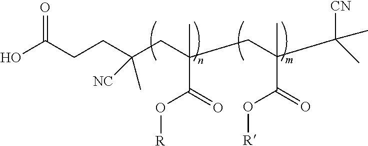

where R has the formula

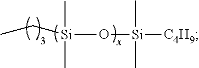

R′ has the formula

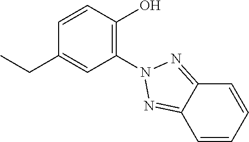

where x is an integer from about 5 to about 10; n is a mole percent from about 70% to about 95%; and m is a mole percent from about 5% to about 30%.

In one or more embodiments, the bottlebrush polymer of the present invention includes any one or more of the above referenced embodiments of the first aspect of the present invention wherein the bottlebrush polymer is optically clear.

In a second aspect, the present invention is directed to a filling material for use in an artificial lens comprising one or more optically clear bottlebrush polymers having a refractive index of from about 1.39 to about 1.48, preferably from about 1.40 to about 1.46, and more preferably from about 1.42 to about 1.46 and a complex viscosity of from about 0.5 to about 50 Pa.s. In some embodiments, the artificial lens is an accommodating intraocular lens (A-IOL) or a presbyopia-correcting IOL.

In one or more embodiments, the filling material of the present invention includes any one or more of the above referenced embodiments of the second aspect of the present invention wherein the one or more optically clear bottlebrush polymer is a homopolymer of a methacrylate macromolecule monomer selected from the group consisting of monomethacryloxypropyl terminated polydimethylsiloxane, asymmetric (PDMS-MA) and oligo(ethylene glycol) methacrylate (OEGMA) or a copolymer of at least one of PDMS-MA and OEGMA and at least one methacrylate or acrylate monomer selected from the group consisting of 2,2,2-trifluoroethyl methacrylate (TFEMA), 3,3,4,4,5,5,6,6,7,7,8,8,9,9,10,10,10-heptadecafluorodecyl methacrylate (HDFDMA), benzyl methacrylate (BzMA), 2-[3-(2H-Benzotriazol-2-yl)-4-hydroxyphenyl]ethyl methacrylate (BzTAzMA), ethyleneglycol phenylether methacrylate (EGPhEMA), hydroxyethyl methacrylate (HEMA), 2,2,2-trifluoroethyl acrylate (TFEA), 3,3,4,4,5,5,6,6,7,7,8,8,9,9,10,10,10-heptadecafluorodecyl acrylate (HDFDA), benzyl acrylate (BzA), 2-[3-(2H-Benzotriazol-2-yl)-4-hydroxyphenyl]ethyl acrylate (BzTAzA), ethyleneglycol phenylether acrylate (EGPhEA), hydroxyethyl acrylate (HEA), and combinations thereof, and having one or more end groups derived from a reversible addition fragmentation chain-transfer (RAFT) agent. In one or more embodiments, the filling material of the present invention includes any one or more of the above referenced embodiments of the second aspect of the present invention wherein the optically clear bottlebrush polymer is a copolymer of monomethacryloxypropyl terminated polydimethylsiloxane, asymmetric (PDMS-MA) and oligo(ethylene glycol) methacrylate (OEGMA) comprising from about 10 to about 95 mole percent, preferably from about 10 to 90 mole percent, and more preferably from about 10 to about 80 mole percent PDMS-MA.

In one or more embodiments, the filling material of the present invention includes any one or more of the above referenced embodiments of the second aspect of the present invention wherein the optically clear bottlebrush polymer has a complex viscosity of from about 0.5 to about 30 Pa·s at 37° C. In one or more embodiments, the filling material of the present invention includes any one or more of the above referenced embodiments of the second aspect of the present invention wherein the optically clear bottlebrush polymer has a refractive index of from about 1.39 to about 1.48, preferably from about 1.40 to about 1.46, and more preferably from about 1.42 to about 1.46 at 37° C.

In one or more embodiments, the filling material of the present invention includes any one or more of the above referenced embodiments of the second aspect of the present invention wherein the RAFT agent is selected from dithiobenzoates, trithiocarbonates, and combinations thereof. In one or more embodiments, the filling material of the present invention includes any one or more of the above referenced embodiments of the second aspect of the present invention wherein the RAFT agent has a formula selected from:

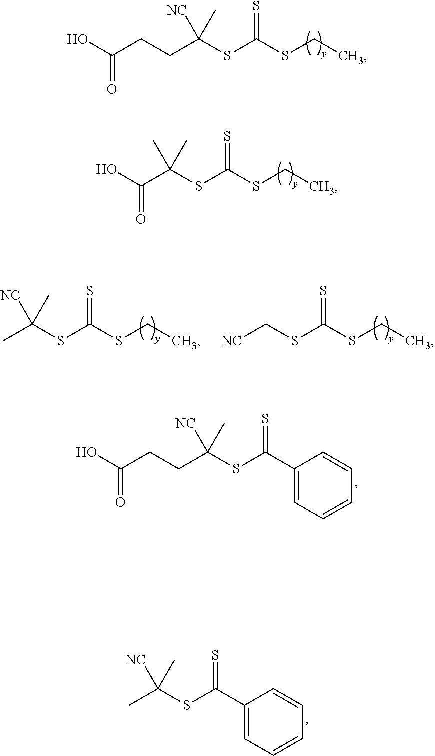

and combinations thereof, where y is an integer from about 3 to about 11.

In one or more embodiments, the filling material of the present invention includes any one or more of the above referenced embodiments of the second aspect of the present invention wherein the optically clear bottlebrush polymer has the formula:

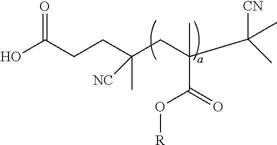

wherein R has the formula

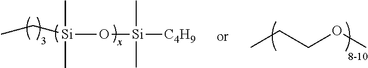

where x is an integer from 5 to 10, and a is an integer from about 20 to about 300.

In one or more embodiments, the filling material of the present invention includes any one or more of the above referenced embodiments of the second aspect of the present invention wherein the optically clear bottlebrush polymer has the formula:

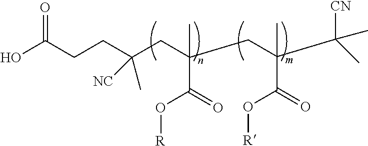

where R has the formula

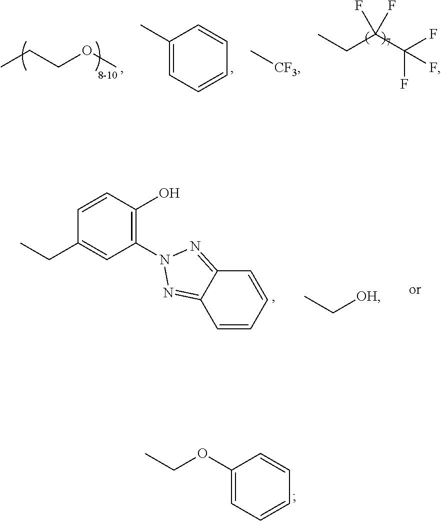

R′ has the formula

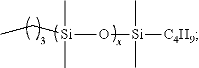

n is an mole percent from about 5% to about 30%; m is a mole percent from about 70% to about 95%; and y is an integer from about 5 to about 10.

In a third aspect, the present invention is directed to an intraocular lens comprising: a filling medium and a capsular interface configured and dimensioned to be received within the natural eye capsule, and to be filled with the filling medium either prior to insertion in the eye or in situ, wherein the filling material comprises one or more optically clear bottlebrush polymers having a refractive index from about 1.39 to about 1.48, preferably from about 1.40 to about 1.46, and more preferably from about 1.42 to about 1.46 and a complex viscosity from about 0.5 Pa.s to about 50 Pa.s, wherein the capsular interface filled with the filling medium defines a predetermined optical power. In one or more embodiments, the capsular interface filled with the filling medium is an accommodating lens that responds to the action of the ciliary muscles and adjusts to an altered shape.

In one or more embodiments, the intraocular lens of the present invention includes any one or more of the above referenced embodiments of the third aspect of the present invention wherein the capsular interface and the filling medium defines a first optical power and, in response to the action of the ciliary muscle, alters its shape to define a second optical power. In one or more embodiments, the intraocular lens of the present invention includes any one or more of the above referenced embodiments of the third aspect of the present invention wherein the first and second optical powers are predetermined by at least the shape and refractive index of the capsular interface, and the refractive index of the filling medium, such that the first and second optical powers vary depending on the shape and refractive index of the capsular interface, and the refractive index of the filling medium.

In one or more embodiments, the intraocular lens of the present invention includes any one or more of the above referenced embodiments of the third aspect of the present invention wherein the surface of the capsular interface is coated with ocular medications or with substances used to prevent the formation of posterior capsular opacification (PCO)

In one or more embodiments, the intraocular lens of the present invention includes any one or more of the above referenced embodiments of the third aspect of the present invention wherein the capsular interface when filled comes in a range of predetermined dimensions from about 9 mm to 11 mm in diameter to about 4-6 mm thick, depending on the size of the patient's capsular bag. In one or more embodiments, the intraocular lens of the present invention includes any one or more of the above referenced embodiments of the third aspect of the present invention which corrects for corneal astigmatism through different powers built along different meridians of the polymeric capsule interface or through filling medium with different refractive indexes in different compartments within the intraocular lens.

In various embodiments, the intraocular lens of the present invention includes any one or more of the above referenced embodiments of the third aspect of the present invention wherein the optically clear bottlebrush polymer is a homopolymer of a methacrylate macromolecule monomer selected from the group consisting of monomethacryloxypropyl terminated polydimethylsiloxane, asymmetric (PDMS-MA) and oligo(ethylene glycol) methacrylate (OEGMA) or a copolymer of at least one of PDMS-MA and OEGMA and at least one methacrylate or acrylate monomer selected from the group consisting of a, 2,2,2-trifluoroethyl methacrylate (TFEMA), 3,3,4,4,5,5,6,6,7,7,8,8,9,9,10,10,10-heptadecafluorodecyl methacrylate (HDFDMA), benzyl methacrylate (BzMA), 2-[3-(2H-Benzotriazol-2-yl)-4-hydroxyphenyl]ethyl methacrylate (BzTAzMA), ethyleneglycol phenylether methacrylate (EGPhEMA), hydroxyethyl methacrylate (HEMA), 2,2,2-trifluorocthyl acrylate (TFEA), 3,3,4,4,5,5,6,6,7,7,8,8,9,9,10,10,10-heptadecafluorodecyl acrylate (HDFDA), benzyl acrylate (BzA), 2-[3-(2H-Benzotriazol-2-yl)-4-hydroxyphenyl]ethyl acrylate (BzTAZA), ethyleneglycol phenylether acrylate (EGPhEA), hydroxyethyl acrylate (HEA), and combinations thereof, and having one or more end groups derived from a reversible addition fragmentation chain-transfer (RAFT) agent. In one or more embodiments, the intraocular lens of the present invention includes any one or more of the above referenced embodiments of the third aspect of the present invention wherein the optically clear bottlebrush polymer is a copolymer of monomethacryloxypropyl terminated polydimethylsiloxane, asymmetric (PDMS-MA) and oligo(ethylene glycol) methyl ether methacrylate (OEGMA) comprising from about 10 to about 95 mole percent PDMS-MA.

In one or more embodiments, the intraocular lens of the present invention includes any one or more of the above referenced embodiments of the third aspect of the present invention wherein the optically clear bottlebrush polymer comprises the residue of an ultra-violet (UV) light blocking methacrylate monomer. In one or more embodiments, the intraocular lens of the present invention includes any one or more of the above referenced embodiments of the third aspect of the present invention wherein the ultra-violet (UV) light blocking methacrylate monomer is 2-[3-(2H-Benzotriazol-2-yl)-4-hydroxyphenyl]ethyl methacrylate (BzTAzMA).

In one or more embodiments, the intraocular lens of the present invention includes any one or more of the above referenced embodiments of the third aspect of the present invention wherein the clear bottlebrush polymer has a complex viscosity from about 0.5 to about 15 Pa·s. In one or more embodiments, the intraocular lens of the present invention includes any one or more of the above referenced embodiments of the third aspect of the present invention wherein the optically clear bottlebrush polymer has a refractive index from about 1.43 to about 1.48.

In one or more embodiments, the intraocular lens of the present invention includes any one or more of the above referenced embodiments of the third aspect of the present invention wherein the optically clear bottlebrush polymer has the formula:

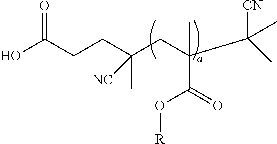

where R has the formula

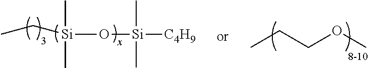

where x is an integer from about 5 to about 10; and a is an integer from about 20 to about 300. In various other embodiments, the intraocular lens of the present invention includes any one or more of the above referenced embodiments of the third aspect of the present invention wherein the optically clear bottlebrush polymer has the formula:

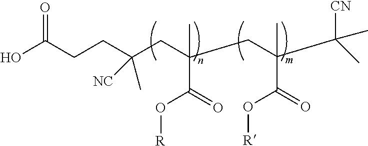

wherein R has the formula

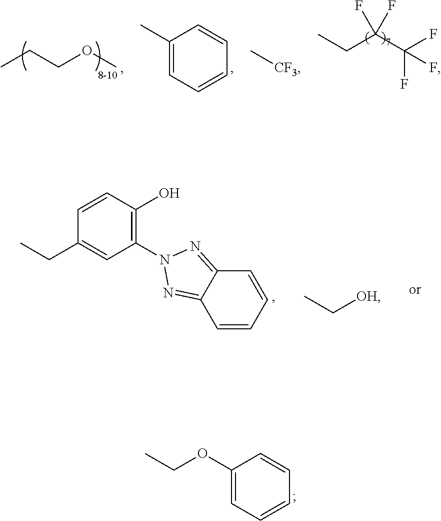

R′ has the formula

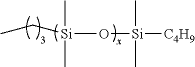

where x is an integer from 5 to 10; n is a mole percent from about 5% to about 30%; m is a mole percent from about 70% to about 95%.

In some embodiments, the intraocular lens of the present invention includes any one or more of the above referenced embodiments of the third aspect of the present invention wherein the optically clear bottlebrush polymer has the formula:

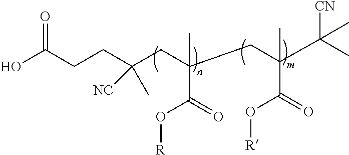

wherein R has the formula

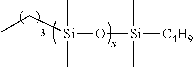

and R′ has the formula

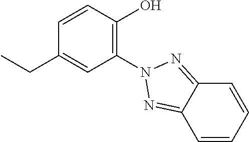

where x is an integer from 5 to 10; n is a mole percent from about 5% to about 30%; and m is a mole percent from about 70% to about 95%.

These and other features of the systems and methods of the subject invention will become more readily apparent to those skilled in the art from the following detailed description of the preferred embodiments taken in conjunction with the drawings.

### BRIEF DESCRIPTION OF THE DRAWINGS

For a more complete understanding of the features and advantages of the present invention, reference is now made to the detailed description of the invention along with the accompanying figures in which:

FIG. 1 is an image of an optically clear poly(PDMS-MA) bottlebrush polymer after end-group removal.

FIG. 2 is a schematic diagram of an IOL comprising a thin flexible shell 2a filled with the optically clear filling medium 2b.

FIG. 3 is a schematic diagram of an IOL showing the thin flexible shell of the IOL 3a inserted into the capsular bag 3f with an inserter/filling device 3b.

FIG. 4 is an image of one version of the A-IOL in which the polymeric shell material is of uniform thickness.

FIG. 5 is a ¹H NMR spectrum of poly(PDMS-MA) in CDCl₃.

FIG. 6 is a ¹H NMR spectrum of poly(OEGMA) in CDCl₃.

FIG. 7 is a ¹H NMR spectrum of poly(PDMS-MA-co-OEGMA) in CDCl₃. (Feed ratio: 70 mol % PDMS-MA and 30 mol % OEGMA. Actual composition: 68 mol % PDMS-MA and 32 mol % OEGMA).

FIG. 8 a ¹H NMR spectrum of poly(PDMS-MA-co-OEGMA) in CDCl₃. (Feed ratio: 90 mol % PDMS-MA and 10 mol % OEGMA. Actual composition: 84 mol % PDMS-MA and 16 mol % OEGMA).

FIG. 9 is a ¹H NMR spectrum of poly(PDMS-MA-co-OEGMA) in CDCl₃. (Feed ratio: 10 mol % PDMS-MA and 90 mol % OEGMA. Actual composition: 20 mol % PDMS-MA and 80 mol % OEGMA).

FIG. 10 is a ¹H NMR spectrum of poly(PDMS-MA-co-BzMA) in CDCl₃. (Feed ratio: 70 mol % PDMS-MA and 30 mol % BzMA. Actual composition: 58 mol % PDMS-MA and 42 mol % BzMA).

FIG. 11 is a ¹H NMR spectrum of poly(PDMS-MA-co-BzMA) in CD₂Cl₂. (Feed ratio: 90 mol % PDMS-MA and 10 mol % BzMA. Actual composition: 80 mol % PDMS-MA and 20 mol % BzMA).

FIG. 12 is a ¹H NMR spectrum of poly(PDMS-MA-co-EGPhEMA) in CDCl₃. (Feed ratio: 70 mol % PDMS-MA and 30 mol % EGPhEMA. Actual composition: 64 mol % PDMS-MA and 36 mol % EGPhEMA).

FIG. 13 is a ¹H NMR spectrum of poly(OEGMA-co-EGPhEMA) in CD₂Cl₂. (Feed ratio: 90 mol % PDMS-MA and 10 mol % EGPhEMA. Actual composition: 81 mol % OEGMA and 19 mol % EGPhEMA).

FIG. 14 is a ¹H NMR spectrum of poly(PDMS-MA-co-TFEMA) in CDCl₃. (Feed ratio: 70 mol % PDMS-MA and 30 mol % TFEMA. Actual composition: 66 mol % PDMS-MA and 34 mol % TFEMA).

FIG. 15 is a ¹H NMR spectrum of poly(PDMS-MA-co-TFEMA) in CDCl₃. (Feed ratio: 50 mol % PDMS-MA and 50 mol % TFEMA. Actual composition: 48 mol % PDMS-MA and 52 mol % TFEMA).

FIG. 16 is a ¹H NMR spectrum of poly(PDMS-MA-co-BzTAzMA) in CDCl₃.

FIG. 17 shows THF SEC traces of poly(PDMS-MA) with three different molecular weights. (a: Mₙ=13,370 g/mol, b: Mₙ=27,160 g/mol, c: Mₙ=67,900 g/mol).

FIG. 18 shows THE SEC traces of poly(PDMS-MA) with three detectors, top trace: refractive index detector, middle trace: UV detector at 254 nm, bottom trace: light scattering detector

FIG. 19 is a graph showing complex viscosity measurements of poly(PDMS-MA), POEGMA, and their random copolymers with various ratios at 25° C.

FIG. 20 is a graph showing complex viscosity measurements of poly(PDMS-MA), poly(PDMS-MA₇₀-co-BzMA₃₀), and poly(PDMS-MA₇₀-co-EGPhEMA₃₀) at 25° C.

FIG. 21 is a graph showing complex viscosity measurements of poly(PDMS-MA₉₀-co-BzMA₁₀) and poly(OEGMA₉₀-co-EGPhEMA₁₀) at 25° C.

FIG. 22 is a graph showing complex viscosity measurements of poly(PDMS-MA) at various temperatures (M_{n.theo}=30,000-40,000 g/mol).

FIG. 23 is a graph showing complex viscosity measurements of poly(PDMS-MA) at various temperatures (M_{n.theo}>200,000 g/mol).

FIG. 24 is a graph comparing refractive index (RI) and viscosity for polydimethylsiloxane methacrylate (PDMS-MA), heptadecafluorodecyl methacrylate (HDFDMA), trifluoroethyl methacrylate (TFEMA), oligoethyleneglycol methacrylate (OEGMA), Benzyl methacrylate (BzMA), and ethylene glycol phenyl ether methacrylate (EGPhEMA).

### DETAILED DESCRIPTION OF THE ILLUSTRATIVE EMBODIMENTS

The following is a detailed description of the disclosure provided to aid those skilled in the art in practicing the present disclosure. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description of the disclosure herein is for describing particular embodiments only and is not intended to be limiting of the disclosure.

In one or more embodiments, the present invention provides a synthetic route to generating biostable polymer bottlebrushes that have tunable viscosity and optical properties making them well suited for use as an optically clear filing fluid for intraocular lenses. The method controls with precision, the refractive index of the bottlebrush and the viscosity properties necessary for the fluid to fit into the lens and avoids the use of solvents or other diluting fluids. In some embodiments, RAFT polymerization techniques are used to make clear BBPs for use in artificial lenses for treatment of cataracts. In one or more of these embodiments, two macromonomers with low glass transition temperatures (T_{g}), different refractive indices (nᵣ), and hydrophobicity are used. In some of these embodiments, high refractive index methacrylate macromonomers like poly(dimethylsiloxane)-methacrylate (PDMS-MA) and/or oligo(ethylene glycol) methacrylate (OEGMA) may be used to form the BBPs. PDMS-MA is a hydrophobic macromonomer with nᵣ of 1.41-142. Oligo(ethylene glycol) methacrylate (OEGMA), on the other hand, is a hydrophilic macromonomer with nᵣ of 1.45-1.46. In some embodiments, these two macromonomers can be homopolymerized to yield poly(PDMS-MA) and poly(OEGMA) or copolymerized to yield poly(PDMS-MA-random-OEGMA) all of which are honey-like viscous liquids with a complex viscosity in the range of 0.4-12 Pa·s. It has also been found that low and high nᵣ small molecule monomers can be copolymerized with PDMS-MA and OEGMA to tune the final nᵣ without increasing the viscosity outside of the range. So, in some embodiments, fluorinated methacrylic monomers are copolymerized with PDMS-MA to decrease the nᵣ and benzylic monomers are copolymerized with OEGMA to reach the upper limit of nᵣ. In these embodiments, the final nᵣ range is 1.40-1.48. Additionally, it has been found that using the similar copolymerization approach, it is possible to incorporate a UV-absorbing reagent into the BBP backbone to filter out UV light by meth(acrylating) a UV adsorbing dye containing an alcohol or amine group to the bottle brush in or at the end of a polymerization reaction.

The following terms may have meanings ascribed to them below, unless specified otherwise. As used herein, the terms “comprising” “to comprise” and the like do not exclude the presence of further elements or steps in addition to those listed in a claim. Similarly, the terms “a,” “an” or “the” before an element or feature does not exclude the presence of a plurality of these elements or features, unless the context clearly dictates otherwise.

The phrase “and/or.” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, the terms “comprising” “to comprise” and the like, are intended to be open ended and do not exclude the presence of further elements or steps in addition to those listed in a claim or other sentence. For example, a polymer is the to comprise a specific type of linkage if that linkage is present in the polymer, even if other linkages are also present.

Throughout this application, the term “about” is used to indicate that a value includes the inherent variation of error for the device, or the method being employed to determine the value, or the variation that exists among the samples being measured. Unless otherwise stated or otherwise evident from the context, the term “about” means within 10% (i.e., within 10%, 9%, 8%, 7%, 6%, 5%, 4%, 3%, 2%, 1%, or less) above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents. Unless otherwise clear from context, all numerical values provided herein in the specification and the claim can be modified by the term “about.”

It should also be understood that the ranges provided herein are a shorthand for all the values within the range and, further, that the individual range values presented herein can be combined to form additional non-disclosed ranges. For example, a range of 1 to 50 is understood to include not only 1 and 50, but any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

A polymer “comprises” or is “derived from” a stated monomer if that monomer is incorporated into the polymer. Thus, the incorporated monomer that the polymer comprises is not the same as the monomer prior to incorporation into a polymer, in that at the very least, certain terminal groups are incorporated into the polymer backbone. A polymer is the to comprise a specific type of linkage if that linkage is present in the polymer.

As will be apparent, the term “polymer” is used to refer to a macromolecule having a series of repeated monomer units or, more broadly, to a material made therefrom. Unless otherwise indicated or otherwise clear from the context, the term “polymer” is intended to be interpreted broadly and encompasses all types of polymers, including, but not limited to, homopolymers, copolymers, block copolymers, random copolymers, and other known polymer species. As used herein, the term “homopolymer” refers to a polymer derived from a single monomeric species. And as follows, unless otherwise indicated, the term “copolymer” refers to a polymer derived from two, three or more monomeric species and includes alternating copolymers, periodic copolymers, random copolymers, statistical copolymers and block copolymers. Unless otherwise indicated, the term “block copolymer” comprises two or more homopolymer or copolymer subunits linked by covalent bonds.

As used herein, the term “residue(s)” is used to refer generally to the part of a monomer or other chemical unit that has been incorporated into a polymer or large molecule. By extension, the terms “residue of the chain transfer agent” and the “chain transfer agent residue” are used interchangeably to refer to the parts of the chain transfer agent that have been incorporated into the bottlebrush polymers. Conversely, a polymer “comprises” or is “derived from” a stated monomer if that monomer is incorporated into the polymer. Thus, the incorporated monomer that the polymer comprises is not the same as the monomer prior to incorporation into a polymer, in that at the very least, certain terminal groups are incorporated into the polymer backbone. A polymer is said to comprise a specific type of linkage if that linkage is present in the polymer.

The term “ultra-violet light” is used herein to refer generally to light in the ultraviolet portion of the spectrum generally having a wavelength of from about 10 nm to about 400 nm. Similarly, the term “ultra-violet light blocking” as applied to a polymer or other material, refers broadly to the ability that polymer or material to block or reduce transmission of ultra-violet light or to a polymer or other material having that ability. A polymer is understood to be “clear” or “transparent” if it is not cloudy and images can be seen through the material. However, a “clear” or “transparent” polymer may be still have a colored “tint,” provided that it does not appear cloudy and images can be seen through the material. The term “optically clear” as applied herein to a polymer refers to a polymer that is “clear,” substantially untinted, and suitable for use in optical applications.

All publications, patent applications, patents, and other references mentioned herein are expressly incorporated by reference in their entirety, which means that they should be read and considered by the reader as part of this text. That the document, reference, patent application, or patent cited in this text is not repeated in this text is merely for reasons of conciseness. In the case of conflict, the present disclosure, including definitions, will control. All technical and scientific terms used herein have the same meaning.

Further, any compositions or methods provided herein can be combined with one or more of any of the other compositions and methods provided herein. The fact that given features, elements, or components are cited in different dependent claims does not exclude that at least some of these features, elements, or components maybe used in combination together.

In a first aspect, the present invention is directed to a bottlebrush homopolymer or copolymer for use as a fluid-like filler material in ocular implants comprising a homopolymer or copolymer of one or more high refractive index methacrylate monomers or macromonomer and a reversible addition-fragmentation chain-transfer (RAFT) agent. The bottlebrush polymers are biocompatible, transparent, and preferably optically clear. As set forth above, these high refractive index methacrylate monomers and macromonomers will all have a terminal reactive methacrylate group capable of RAFT polymerization and a chain length sufficient to provide a polymer having a grafting density high enough to provide rigidity and prevent chain entanglement.

In various embodiments, optically clear bottlebrush polymers of the present invention will be a homopolymer of a high refractive index (1.43 to about 1.48) methacrylate macromonomer having a relatively low glass transition temperature (T_{g}) (−50° C. to about 30° C.) and will have a number average molecular mass (Mₙ) between about 10,000 g/mol and 250,000 g/mol. As used herein, the term “methacrylate macromolecule” refers to a macromolecule having a terminal methacrylate functional group capable of RAFT, Atom Transfer Radical Polymerization (ATRP), ring-opening metathesis polymerization (ROMP), and ring-opening polymerization (ROP) polymerization for form a bottlebrush or comb polymer. Suitable high refractive index methacrylate macromonomers may include, without limitation, monomethacryloxypropyl terminated polydimethylsiloxane (PDMS-MA) and oligo(ethylene glycol) methacrylate (OEGMA).

In some embodiments, the clear bottlebrush polymers of the present invention will be a homopolymer of PDMS-MA having a number average molecular mass (Mₙ) of from about 25,000 g/mole to about 250,000 g/mole, a refractive index (nᵣ) of from about 1.43 to about 1.48, and a complex viscosity of from about 0.5 Pa·s to about 15 Pa·s. In some other embodiments, clear bottlebrush polymers of the present invention will be a homopolymer of OEGMA having a number molecular mass (Mₙ) of from about 25,000 g/mole to about 250,000 g/mole, a nᵣ of from about 1.43 to about 1.48, and a complex viscosity of from about 0.5 Pa·s to about 15 Pa·s.

In one or more embodiment, the clear bottlebrush polymer is a random copolymer of PDMS-MA and OEGMA formed by RAFT polymerization (“poly(PDMS-MA-co-OEGMA)”) and comprises from about 10 to about 95 mole percent PDMS-MA. In some embodiments, the poly(PDMS-MA-co-OEGMA) will comprise from about 20 to about 95, in other embodiments, from about 30 to about 95, in other embodiments, from about 50 to about 95, in other embodiments, from about 70 to about 95, in other embodiments, from about 80 to about 95, in other embodiments, from about 10 to about 85, in other embodiments, from about 10 to about 70, in other embodiments, from about 10 to about 60, in other embodiments, from about 10 to about 50, and in other embodiments, from about 10 to about 30 mole percent PDMS-MA repeat units. In some embodiments, the poly(PDMS-MA-co-OEGMA) will comprise from about 70 to about 95 mole percent PDMS-MA repeat units.

In one or more embodiments, these poly(PDMS-MA-co-OEGMA) copolymers will have a number molecular weight (Mₙ) of from about 25,000 g/mole to about 250,000 g/mole, as measured by size exclusion chromatography (SEC). In some embodiments, the poly(PDMS-MA-co-OEGMA) copolymers will have a number molecular weight (Mₙ) of from about 30,000 g/mole to about 250,000 g/mole, in other embodiments, from about 50,000 g/mole to about 25,000 g/mole, in other embodiments, from about 100,000 g/mole to about 250,000 g/mole, in other embodiments, from about 150,000 g/mole to about 250,000 g/mole, in other embodiments, from about 200,000 g/mole to about 250,000 g/mole, in other embodiments, from about 25,000 g/mole to about 200,000 g/mole, in other embodiments, from about 25,000 g/mole to about 150,000 g/mole, in other embodiments, from about 25,000 g/mole to about 100,000 g/mole, and in other embodiments, from about 25,000 g/mole to about 50,000 g/mole, as measured by size exclusion chromatography (SEC).

In some embodiments, these poly(PDMS-MA-co-OEGMA) copolymers will have a nᵣ of from about 1.40 to about 1.48. In some embodiments, nᵣ will be from about 1.41 to about 1.48, in other embodiments, from about 1.42 to about 1.48, in other embodiments, from about 1.45 to about 1.48, in other embodiments, from about 1.46 to about 1.48, in other embodiments, from about 1.40 to about 1.47, in other embodiments, from about 1.40 to about 1.46, in other embodiments, from about 1.40 to about 1.45, in other embodiments, from about 1.40 to about 1.44, in other embodiments, from about 1.40 to about 1.43, and in other embodiments, from about 1.40 to about 1.42. In one or more of these embodiments, the poly(PDMS-MA-co-OEGMA) copolymer will have a complex viscosity of from about 0.5 Pa·s to about 15 Pa·s, as measured by shear rheology.

In some other embodiments, the PDMS-MA and/or OEGMA or other high refractive index methacrylate macromonomers may be copolymerized with a low and high nᵣ small molecule methacrylate or acrylate monomers to tune the final nᵣ, without increasing the viscosity outside of the desired range. Suitable small molecule methacrylate monomers may include, without limitation, 2,2,2-trifluoroethyl methacrylate (TFEMA), 3,3,4,4,5,5,6,6,7,7,8,8,9,9,10,10,10-heptadecafluorodecyl methacrylate (HDFDMA), benzyl methacrylate (BzMA), 2-[3-(2H-Benzotriazol-2-yl)-4-hydroxyphenyl]ethyl methacrylate (BzTAzMA), ethyleneglycol phenylether methacrylate (EGPhEMA), hydroxyethyl methacrylate (HEMA), 2,2,2-trifluoroethyl acrylate (TFEA), 3,3,4,4,5,5,6,6,7,7,8,8,9,9,10,10,10-heptadecafluorodecyl acrylate (HDFDA), benzyl acrylate (BzA), 2-[3-(2H-Benzotriazol-2-yl)-4-hydroxyphenyl]ethyl acrylate (BzTAZA), ethyleneglycol phenylether acrylate (EGPhEA), hydroxyethyl acrylate (HEA), and combinations thereof. In some embodiments, fluorinated methacrylic monomers are copolymerized with PDMS-MA to decrease the nᵣ. In some other embodiments, benzylic monomers are copolymerized with OEGMA to reach the upper limit of a desired nᵣ range.

In some other embodiments, the clear bottlebrush polymer or copolymer of the present invention is a copolymer of PDMS-MA and benzyl methacrylate (BzMA) formed by RAFT polymerization (“poly(PDMS-MA-co-BzMA)”) and comprising from about 10 to about 90 mole percent PDMS-MA. In some embodiments, the poly(PDMS-MA-co-BzMA) copolymer will comprise from about 20 to about 90, in other embodiments, from about 30 to about 90, in other embodiments, from about 50 to about 90, in other embodiments, from about 70 to about 90, in other embodiments, from about 80 to about 90, in other embodiments, from about 10 to about 85, in other embodiments, from about 10 to about 70, in other embodiments, from about 10 to about 60, in other embodiments, from about 10 to about 50, and in other embodiments, from about 10 to about 30 mole percent PDMS-MA repeat units.

In some other embodiments, the clear bottlebrush polymer is a copolymer of PDMS-MA and ethyleneglycol phenylether methacrylate (EGPhEMA) formed by RAFT polymerization (“poly(PDMS-MA-co-EGPhEMA)”) and comprising from about 10 to about 90 mole percent PDMS-MA. In some embodiments, the poly(PDMS-MA-co-EGPhEMA) copolymer will comprise from about 20 to about 90, in other embodiments, from about 30 to about 90, in other embodiments, from about 50 to about 90, in other embodiments, from about 70 to about 90, in other embodiments, from about 80 to about 90, in other embodiments, from about 10 to about 85, in other embodiments, from about 10 to about 70, in other embodiments, from about 10 to about 60, in other embodiments, from about 10 to about 50, and in other embodiments, from about 10 to about 30 mole percent PDMS-MA repeat units.

In some embodiments, the high refractive index (meth)acrylate macromonomers (e.g., PDMS-MA and OEGMA) and methacrylate monomers (TFEMA, HDFDMA, BzMA, BzTAzMA, EGPhEMA, and HEMA) may have one of the following formulas:

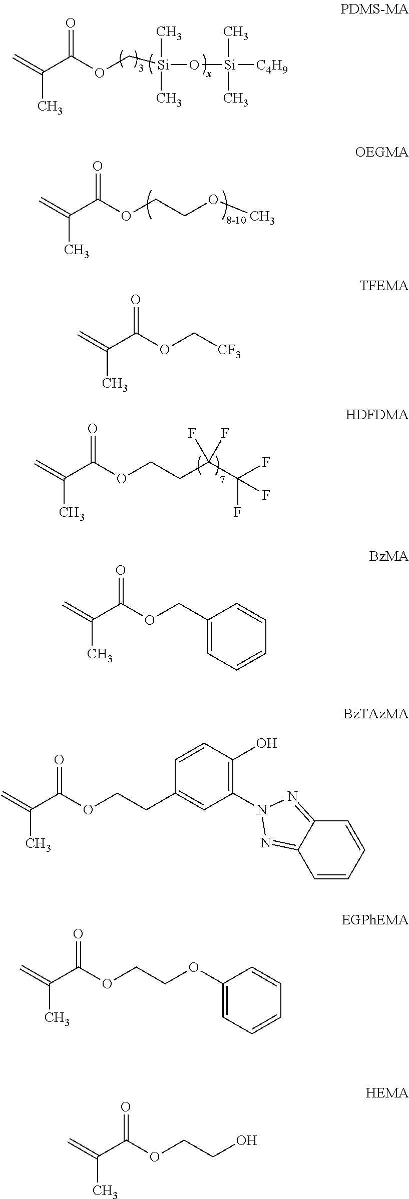

where x is an integer from about 5 to about 10.

In some other embodiments, the acrylate monomers (TFEA, HDFDA, BZA, BzTAZA, EGPhEA, HEA) may have one of the following formulas:

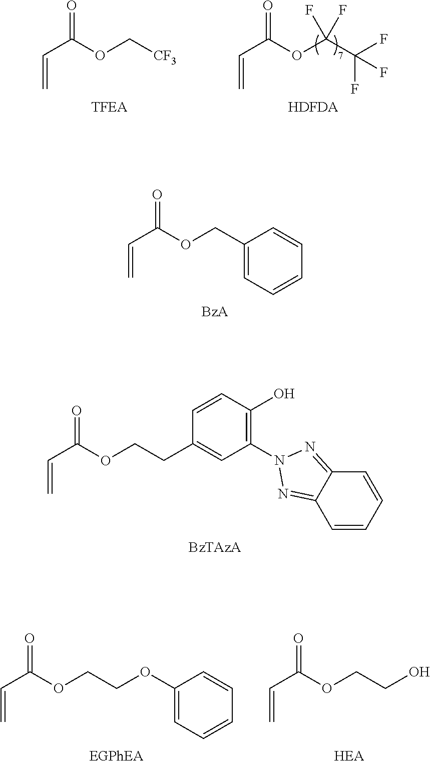

Particularly in embodiments where the clear bottlebrush polymers and copolymers of the present invention are used as a filler material in an intraocular lens (IOL) or accommodating intraocular lens (A-IOL), both the viscosity and the refractive index (nᵣ) are important. In various embodiments, the optically clear bottlebrush polymers of the present invention will have a complex viscosity of from about 0.4 Pa·s to about 15 Pa·s, as measured by rheometer at 37° C. In some embodiments, the optically clear bottlebrush polymers of the present invention will have a complex viscosity of from about 0.5 Pa·s to about 15 Pa·s, in other embodiments, from about 0.5 Pa·s to about 13 Pa·s, in other embodiments, from about 0.5 Pa·s to about 12 Pa·s, in other embodiments, from about 0.5 Pa·s to about 10 Pa·s, in other embodiments, from about 0.5 Pa·s to about 8 Pa·s, in other embodiments, from about 0.5 Pa·s to about 6 Pa·s, in other embodiments, from about 1 Pa·s to about 15 Pa·s, in other embodiments, from about 3 Pa·s to about 15 Pa·s, in other embodiments, from about 5 Pa·s to about 15 Pa·s, in other embodiments, from about 7 Pa·s to about 15 Pa·s, and in other embodiments, from about 9 Pa·s to about 15 Pa·s at 37 ° C. In some embodiments, the optically clear bottlebrush polymers of the present invention will have a complex viscosity of from about 0.4 Pa·s to about 50 Pa·s, as measured by rheometer at 37 ° C.

In one or more embodiment, the optically clear bottlebrush polymers and copolymers of the present invention will have a refractive index of from about 1.43 to about 1.48, as measured by a refractometer at 37° C. In some embodiments, the optically clear bottlebrush polymers and copolymers of the present invention will have a refractive index of from about 1.40 to about 1.48, in other embodiments from about 1.42 to about 1.48, in other embodiments from about 1.44 to about 1.48, in other embodiments from about 1.46 to about 1.48, in other embodiments from about 1.40 to about 1.47, in other embodiments from about 1.40 to about 1.46, in other embodiments from about 1.40 to about 1.45, and in other embodiments from about 1.40 to about 1.43, as measured by refractometer at 37° C. The refractive index of several clear bottlebrush polymers and copolymers according to the present invention are set forth on Table 1, below.

TABLE 1
Refractive indices of (co)polymer brushes and combs.

| Polymer | Refractive Index, nᵣ (T = 37° C.) |
|---|---|
| Poly(PDMS-MA-co-HDFDMA) | 1.41840 |
| Poly(PDMS-MA-co-TFEMA) | 1.42785 |
| Poly(PDMS-MA) | 1.43256 |
| poly(PDMS-MA-co-BzTAzMA) | 1.44041 |
| Poly(PDMS-MA₉₀-co-OEGMA₁₀) | 1.43587 |
| Poly(PDMS-MA₇₀-co-OEGMA₃₀) | 1.43600 |
| Poly(PDMS-MA₁₀-co-OEGMA₉₀) | 1.46250 |
| Poly(OEGMA) | 1.46750 |
| Poly(PDMS-MA₇₀-co-BzMA₃₀) | 1.44745 |
| Poly(PDMS-MA₇₀-co-EGPhEMA₃₀) | 1.44740 |
| Poly(PDMS-MA₉₀-co-BzMA₁₀) | 1.43981 |
| Poly(OEGMA₉₀-co-EGPhEMA₁₀) | 1.47694 |

It has been found that by altering the stoichiometry and composition based on refractive index measurements, it is possible to finely tune the refractive index of the clear bottlebrush polymers and copolymers of the present invention.

The bottlebrush polymers of the present invention may be made by any suitable method but are preferably made using reversible addition-fragmentation chain-transfer (RAFT) polymerization techniques. In various embodiments, the bottlebrush polymers of the present invention may be formed by RAFT polymerization using one or more suitable RAFT agents and a conventional free-radical initiator. Exemplary reaction mechanisms are shown in Schemes 1-11 and are discussed in more depth below.

Suitable RAFT agents may include, without limitation, dithiobenzoates, trithiocarbonates, and combinations thereof. In some of these embodiments, the RAFT agent may be 4-cyano-4-[(dodecylsulfanylthiocarbonyl)sulfanyl] pentanoic acid (chain transfer agent-1, CTA1) or 4-cyano-4-(thiobenzoylthio)pentanoic acid (CTA2). In some embodiments, the RAFT agent may be a dithiobenzoate having the formula:

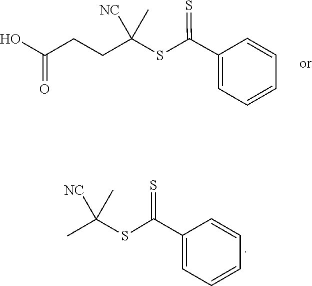 [SMILES: CC(C#N)(CCC(=O)O)SC(=S)c1ccccc1 ; CC(C)(C#N)SC(=S)c1ccccc1]

In some other embodiments, the RAFT agent may be a trithiocarbonates having the formula:

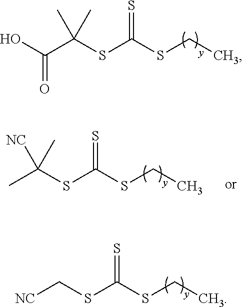

where y is an integer from 3 to 11. In still other embodiments, the RAFT agent will have the formula:

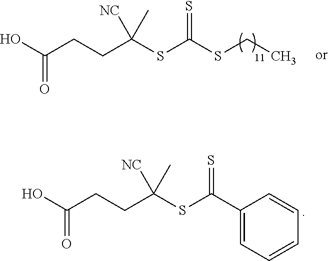

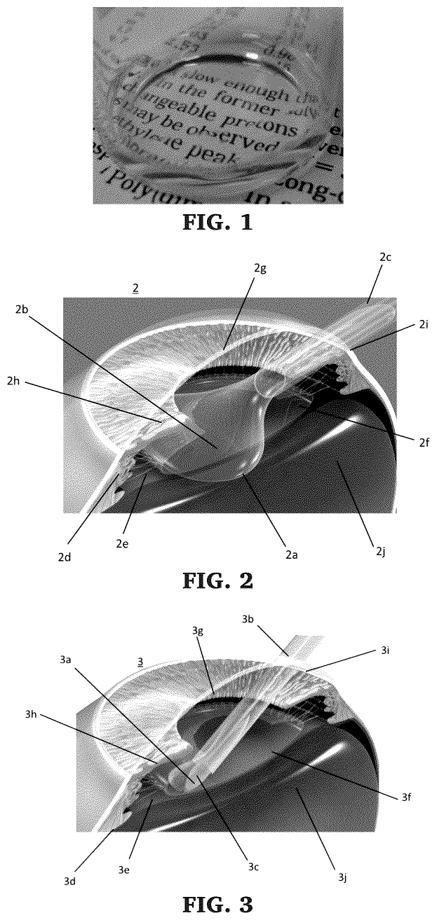

During the RAFT polymerization reaction, the methacrylate macromonomers will polymerize at a location at or near the center of the RAFT agent, splitting the RAFT agent, with each half of the RAFT agent forming an end group for the bottlebrush polymer formed. In embodiments where the bottlebrush polymers of the present invention required to be optically clear, one of these end groups may be removed by the addition of an excess of thermally or chemically activated radical generating compound, such as 2,2′-Azobis(2-methylpropionitrile) (AIBN). The resulting polymers are optically clear, as shown in FIG. 1.

The optically clear bottlebrush polymers of the present invention will have a number average molecular mass Mₙ of from about 25,000 g/mole to about 250,000 g/mole. In some embodiments, the Mₙ of the optically clear bottlebrush polymers of the present invention is from about 50,000 g/mole to about 250,000 g/mole, in other embodiments, from about 100,000 g/mole to about 250,000 g/mole, in other embodiments, from about 150,000 g/mole to about 250,000 g/mole, in other embodiments, from about 200,000 g/mole to about 250,000 g/mole, in other embodiments, from about 25,000 g/mole to about 200,000 g/mole, in other embodiments, from about 25,000 g/mole to about 150,000 g/mole, in other embodiments, from about 25,000 g/mole to about 100,000 g/mole, and in other embodiments, from about 25,000 g/mole to about 50,000 g/mole, as measured by size exclusion chromatography (SEC). In some embodiments, Mₙ is from about 50,000 g/mole to about 200,000 g/mole, as measured by size exclusion chromatography (SEC). In some other embodiments, Mₙ is from about 100,000 g/mole to about 200,000 g/mole, as measured by size exclusion chromatography (SEC). The optically clear bottlebrush polymers of the present invention will have a number average molecular mass (M_{w}) of from about 30,000 g/mole to about 500,000 g/mole, preferably from about 100,000 g/mole to about 400,000 g/mole, and more preferably from about 150,000 g/mole to about 300,000 g/mole, as measured by size exclusion chromatography (SEC).

In various embodiments, the optically clear bottlebrush polymers of the present invention will have a glass transition temperature (T_{g}) of from about −50° C. to about 30° C., preferably from about −50° C. to about 10° C., and more preferably from about −50° C. to about '10° C., as measured by Dynamic Mechanical Analysis (DMA), or Differential Scanning calorimetry (DSC).

In one or more embodiments, the side chain DP of the macromonomers and resulting polymer segments will be from about 5 to 10. In some embodiments, the PDMS-MA macromonomer will have a side chain DP of from about 5 to about 10. In some other embodiments the OEGMA macromonomer will have a side chain DP of from about 8 to about 10.

In one or more embodiments, the bottlebrush polymers of the present invention will have the formula:

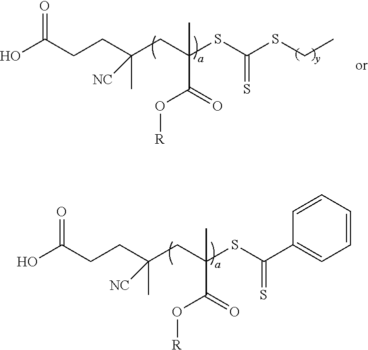

wherein R has the formula

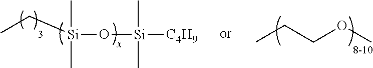

where x is an integer from about 5 to about 10; y is an integer from about 3 to about 11; and a is an integer from about 20 to about 300. In some embodiments, x may be an integer from about 6 to about 10, in other embodiments, from about 7 to about 10, in other embodiments, from about 8 to about 10, in other embodiments, from about 5 to about 9, in other embodiments, from about 5 to about 8, and in other embodiments, from about 5 to about 7. In some embodiments, x is 5. In other embodiments, x is 6.

In some embodiments, y is an integer from about 4 to about 11, in other embodiments, from about 5 to about 11, in other embodiments, from about 6 to about 11, in other embodiments, from about 8 to about 11, in other embodiments, from about 10 to about 11, in other embodiments, from about 3 to about 9, in other embodiments, from about 3 to about 7, and in other embodiments, from about 3 to about 5.In some embodiments, y is 11. In some embodiments, a may be an inter from about 30 to about 300, in other embodiments, from about 50 to about 300, in other embodiments, from about 100 to about 300, in other embodiments, from about 150 to about 300, in other embodiments, from about 200 to about 300, in other embodiments, from about 20 to about 200, in other embodiments, from about 20 to about 100, in other embodiments, from about 20 to about 50, and in other embodiments, from about 20 to about 30.

In one or more embodiments, the bottlebrush polymers of the present invention will have the formula:

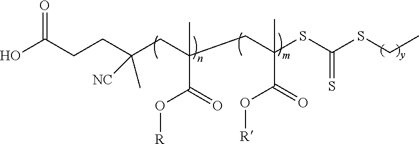

wherein R has the formula

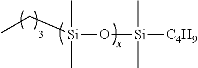

where x is an integer from about 5 to about 10; R′ has the formula

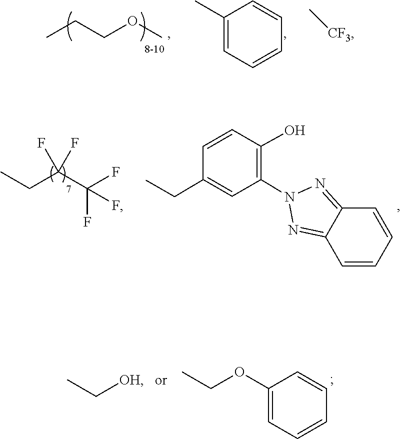

y is an integer from about 3 to about 11; n is a mole percent from about 70% to about 95%; m is a mole percent from about 5% to about 30%; and n+m=100. In various embodiments, x and y may be as set forth above. In some embodiments, y is 11.

In some embodiments, n is a mole percent from about 75% to about 95%, in other embodiments, from about 80% to about 95%, in other embodiments, from about 85% to about 95%, in other embodiments, from about 90% to about 95%, in other embodiments, from about 70% to about 90%, in other embodiments, from about 70% to about 85%, in other embodiments, from about 70% to about 80%, and in other embodiments, from about 70% to about 75%. In various embodiments, m is a mole percent from about 10% to about 30%, in other embodiments, from about 15% to about 30%, in other embodiments, from about 20% to about 30%, and in other embodiments, from about 25% to about 30%.

In one or more embodiments, the bottlebrush polymers of the present invention will have the formula:

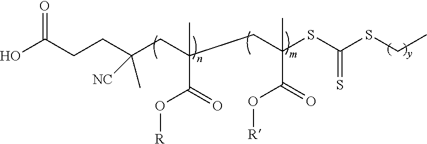

wherein R has the formula

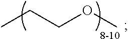

R′ has the formula

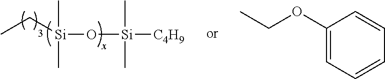

where x is an integer from about 5 to about 10; y is an integer from about 3 to about 11; n is an mole percent from about 80% to about 90%; m is a mole percent from about 20% to about 10%; and n+m=100. In various embodiments, x and y may be any of the integers as set forth above for x and y, and n and m may be any of the mole percent as set forth above for n and m.

In one or more embodiments, the bottlebrush polymers of the present invention will have the formula:

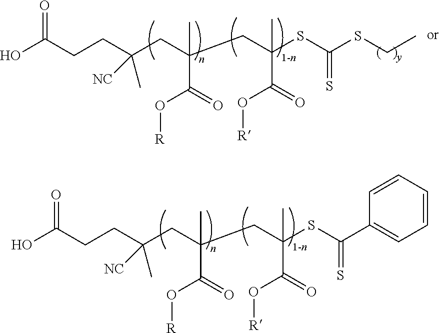

where R has the formula

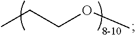

R′ has the formula

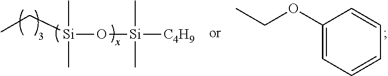

n is a mole percent from about 80% to about 0.90%; y is an integer from about 3 to about 11; and x is an integer from about 5 to about 10. In some embodiments, x is 5 or 6.

In one or more embodiments, the bottlebrush polymers of the present invention will have the formula:

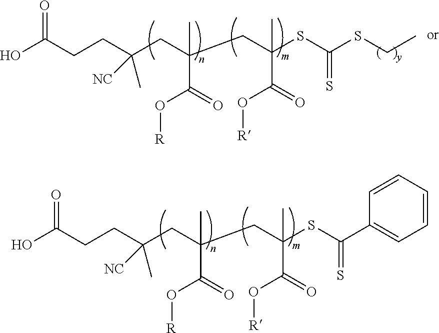

where R has the formula

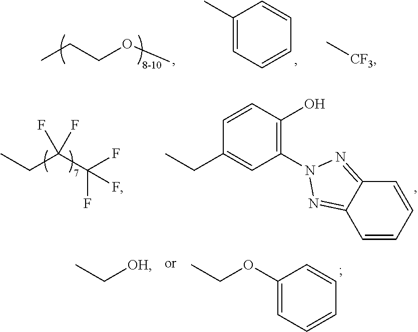

R′ has the formula

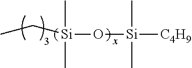

with x being an integer from about 5 to about 10; y is an integer from about 3 to about 11; n is mole percent from about 5% to about 30%; m is a mole percent from about 70% to about 95%; and n+m=100. In some embodiments x is 5 or 6. In some embodiments, y is 11.

While the bottlebrush polymers of the present invention are all transparent after formation by RAFT polymerization, they are often tinted and not fully optically clear. Advantageously, however, it has been found that removing the sulfur containing end group (a residue of the RAFT agent) produces a polymer that is optically clear. (See, e.g., FIG. 1). In one or more embodiments, the bottlebrush polymers of the present invention will be a homopolymer having the formula:

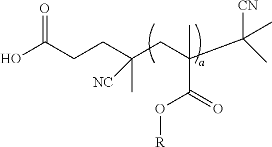

wherein R has the formula

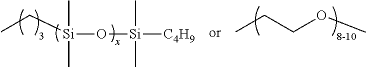

where x is an integer from about 5 to about 10; and a is an integer from about 20 to about 300. In some embodiments, x is 5 or 6.

In some other embodiments, the bottlebrush polymers of the present invention will be a copolymer having the formula:

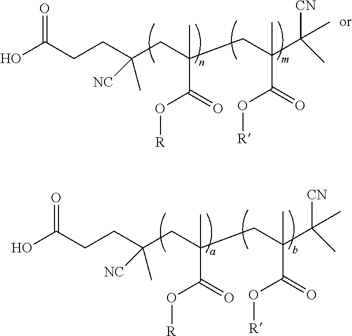

wherein R has the formula

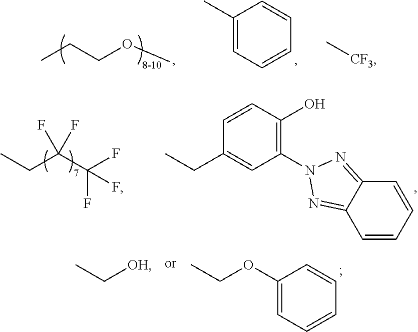

R′ has the formula

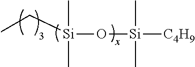

where x is an integer from about 5 to about 10; a is an integer from about 5 to about 30; b is an integer from about 70 to about 95; n is mole percent from about 5% to about 30%; m is a mole percent from about 70% to about 95%; and n+m=100. In some embodiments, x is 5 or 6.

Further, as set forth above, it has been found that using this copolymerization approach it is possible to incorporate a UV-absorbing reagent into the bottle brush polymer backbone to filter out UV light by meth(acrylating) a UV adsorbing dye containing an alcohol or amine group to the bottle brush in or at the end of a polymerization reaction. Suitable UV adsorbing dye containing an alcohol or amine group is 2-[3-(2H-Benzotriazol-2-yl)-4-hydroxyphenyl]ethyl methacrylate (BzTAzMA). In one ore more embodiments, the present invention is directed to a UV light blocking bottlebrush Raft copolymer having the formula:

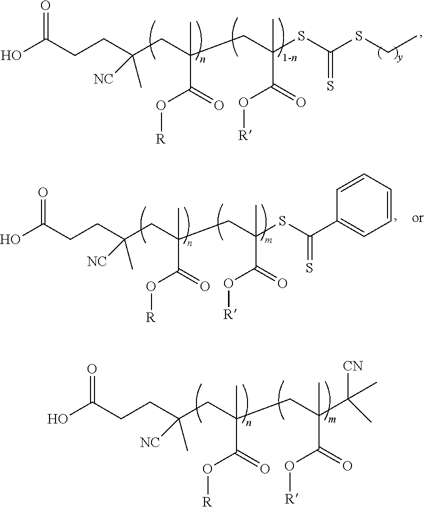

wherein R has the formula

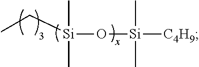

R′ has the formula

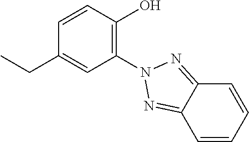

where x is an integer from about 5 to about 10; n is mole percent from about 90% to about 99%; m is a mole percent from about 1% to about 10%; and n+m=100. In some embodiments, x is 5 or 6.

As set forth above, the bottlebrush polymers of the present invention may be made by any suitable method but are preferably made using reversible addition fragmentation chain-transfer (RAFT) polymerization techniques. The clear bottlebrush polymers of the present invention may be made by other techniques including, but not limited to, Atom Transfer Radical Polymerization (ATRP), ring-opening metathesis polymerization (ROMP), and ring-opening polymerization (ROP).

In the embodiments using RAFT polymerization, the one or more high nᵣ methacrylate macromonomer, RAFT agent, and a free-radical initiator are combined in a reaction solvent at an elevated temperature and under an inert atmosphere to produce the bottlebrush polymer. In various embodiments, the one or more high nᵣ methacrylate monomer and/or macromonomer, and the RAFT agent may be any of those described above. The initiator may be any free-radical initiator know in the art, provided that it is nontoxic and compatible with the reagents being used. Suitable initiators may include, without limitation, azo compounds (e.g., 2.2′-azobis(2-methylpropionitrile, AIBN), organic peroxides (e.g., benzoyl peroxide), inorganic peroxides, or combinations thereof.

One of ordinary skill in the art will be able to select a free-radical initiator without undue experimentation. The reaction solvent is not particularly limited provided that capable of dissolving, or at least suspending, all the reagents. Further, since the solvent must be removed, it is preferred that the amount of solvent used be minimized and only enough to dissolve or suspend the other reagents be used. Suitable solvents will include toluene, tetrahydrofuran (THF), hexane, dichloromethane, chloroform, and combinations thereof. One of ordinary skill in the art will be able to select a reaction solvent without undue experimentation. In one or more embodiment, the solvent is toluene.

In some embodiments, bottlebrush polymers of the present invention will be a homopolymer and the molar ratio of macromonomer to RAFT agent to initiator used will be 1-300 eq: 1 eq: 0.5 eq. preferably from 1-200 eq: 1 eq: 0.5 eq, and more preferably 1-100 eq: 1 eq: 0.5 eq. In some embodiments, the molar ratio of macromonomer to RAFT agent to RAFT initiator used will be 100 eq: 1 eq: 0.5 eq.

In various embodiments, the reaction temperature will be between 60° C. and 80° C. preferably between 65° C. and 80° C., and more preferably between 70° C. and 75° C. In some of these embodiments, the reaction temperature is about 70° C. In one or more embodiment, the reaction time will be between 6 hours and 24 hours, preferably between 9 hours and 20 hours, and more preferably between 12 hours and 16 hours. In some of these embodiments, the reaction time will between 12 hours and 16 hours.

A representative reaction mechanism is shown in Scheme 1 below:

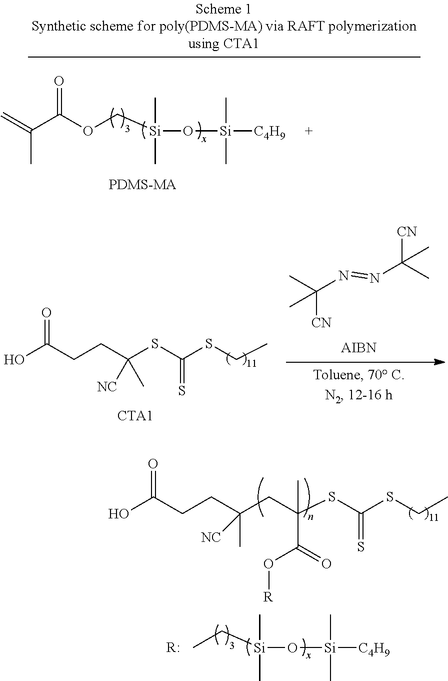

In some of these embodiments, x is an integer from about 5 to about 10, as set forth above. In various embodiments, n is an integer from about 20 to about 300, as set forth above. In the reaction shown in Scheme 1, the high nᵣ methacrylate macromonomer (PDMS-MA) is reacted with the RAFT agent (4-Cyano-4-[(dodecylsulfanylthiocarbonyl)sulfanyl] pentanoic acid (CTA1)) in toluene with an initiator (2,2′-Azobis(2-methylpropionitrile (AIBN)) at about 70 ° C. for from about 12 to about 16 hours to form a poly(PDMS-MA) bottlebrush homopolymer.

In various embodiments, the RAFT polymerization reaction produces a polymer or copolymer having a backbone with a degree of polymerization (DP) as described above. In some embodiments, the polymer or copolymer will have a backbone with a DP of from about 20 to about 300, preferably from about, 50 to about 200, and more preferably from about 75 to 150.

The reaction may be quenched using any suitable method provided that no additional monomers are added to the chain end. In some embodiments, the reaction may be quenched by opening the reaction to ambient air. In some embodiments, the reaction may be quenched by the addition of a weak protic acid in a solvent. In one or more embodiments, the solvent may include, but is not limited to methanol, hexane, heptane, toluene, isopropanol, ethanol, pentane and combinations thereof. In some of these embodiments, reactions including a macromonomer like PDMS-MA are quenched by exposure to the air and the addition of methanol, hexane, heptane, toluene, isopropanol, ethanol, pentane, and combinations thereof. In some other embodiments, reactions including a hydrophillic macromonomer like OEGMA are quenched by exposure to the air and the addition of hexane, heptane, toluene, isopropanol, ethanol, pentane and combinations thereof.

In some other embodiments, controlled radical polymerization (CRP) procedures, including atom transfer radical polymerization and nitroxide medicated Radical polymerization, and ring-opening metathesis polymerization allow the synthesis bottlebrush macromolecules by three different approaches: grafting-onto, grafting-through, and grafting-from. Each is dependent on the use of a monomer that is compatible with the technique. The majority of bottlebrush macromolecules synthesized by ATRP use copper bromide-based catalysts and the grafting-from method. The side chains are polymerized from a macroinitiator which had an initiating group on each monomer unit, resulting in densely grafted polymers with relatively high initiation efficiency and narrow molecular mass distribution without a significant number of inter/intramolecular coupling reactions and retention of the transferable atom at the side-chain end.

The resulting bottlebrush polymers or copolymers may be collected and purified using any suitable method. One of ordinary skill in the art will be able to collect and purify the bottlebrush polymers without undue experimentation. In various embodiments, the bottlebrush polymers or copolymers may be collected and purified as set forth in the Examples below.

As set forth above, while the bottlebrush polymers formed as set forth above are all transparent after their formation by RAFT polymerization, they may be tinted and not fully optically clear. Without wishing to be limited by theory in any way, it is believed that the tinting of these bottlebrush polymers results from the sulfur containing end group (a residue of the RAFT agent, c.f., CTA1,and CTA2). In any event, it has been found that removing these sulfur containing end groups produces a polymer that is optically clear. (See, e.g., FIG. 1).

In embodiments where the bottlebrush polymers of the present invention required to be optically clear (e.g., use in an intraocular lens), these end groups may be removed by the addition of an excess of a thermally or chemically activated radical generating compound, such as 2,2′-Azobis(2-methylpropionitrile) (AIBN). In one or more embodiments, the thermally or chemically activated radical generating compound will be the same compound used to initiate the RAFT polymerization that formed the bottlebrush polymer. In some embodiments, the thermally or chemically activated radical generating compound used to remove the RAFT end groups on the bottlebrush polymers of the present invention will be AIBN.

In one or more of these embodiments, the sulfur containing end-group residue of the chain transfer agent on bottlebrush RAFT polymers of the present invention may be removed at the ω-chain end of poly(PDMS-MA) bottlebrush RAFT polymer and replaced with a 2-cyanopropyl 3⁰ radical end capping group via AIBN treatment as shown in Scheme 2, below.

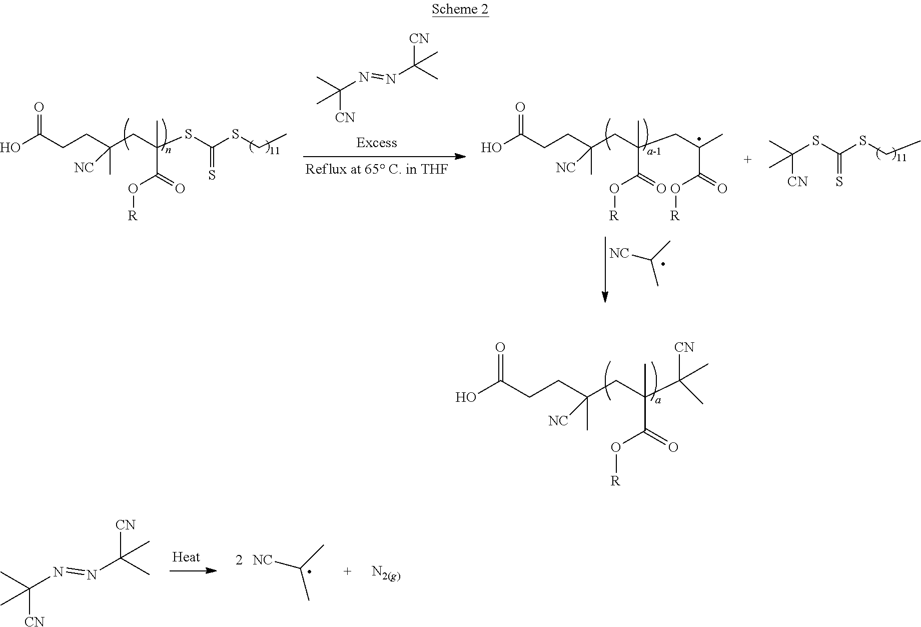

While the bottlebrush polymer shown in Scheme 2 is a homopolymer of PDMS-MA formed using CTA1 as the initiator (see Scheme 1, above), the invention is not to be so limited and the reaction shown in Scheme 2 may also be used with any of the homopolymers and copolymers shown above having a sulfur-containing end group, including those formed with CTA2 as the initiator.

In these embodiments, the bottlebrush polymer is first placed in a sealable flask or other suitable reaction vessel and dissolved with a suitable solvent. The solvent used is not particularly limited and any solvent for the bottlebrush polymers may be used provided that the solvent does not degrade or react with the bottlebrush polymer or other reagents or otherwise interfere with the reaction shown in Scheme 2, above. One of ordinary skill in the art will be able to select a solvent for the bottlebrush polymers without undue experimentation. Suitable solvents may include, without limitation, toluene, tetrahydrofuran (THF), dioxane, dimethylformamide (DMF), and combinations thereof. In some embodiments, the solvent is toluene. In some other embodiments, the solvent is THF.

Next, a thermally or chemically activated radical generating compound is added in excess to the bottlebrush RAFT polymer solution and the vessel is heated to remove the sulfur containing end-group residue of the chain transfer agent from the ω-chain end of bottlebrush RAFT polymer. In the embodiment shown in Scheme 2 above, an excess of AIBN is added to the reaction vessel, which is then sparged with an inert gas, such as nitrogen gas to avoid oxidation of the thiols on the RAFT agent, and then heated to remove the sulfur containing end-group residue from the end of the bottlebrush RAFT polymers. In some embodiments, the reaction vessel may be a flask equipped with a TEFLON™ coated stir bar and may be sealed with a rubber septum.

As will be understood by those of skill in the art, an excess of AIBN will be an amount greater than two times the stoichiometric amount for the polymer. In some embodiments, the AIBN is added in an amount equal to 20 timed the equivalents of RAFT agent used to synthesize the polymer. Once the AIBN has been added, the vessel is sealed and sparged with an inert gas, such as nitrogen gas, to avoid oxidation of the thiols on the RAFT agent. The sparging time is not particularly limited provided it is sufficient to avoid oxidation of the thiols on the RAFT agent and will, of course, depend upon the flow rate of the inert gas used. In some embodiments, the AIBN/bottlebrush RAFT polymer mixture may be sparged with N₂ for 20-30 min (>1 mL/min).

As set for above, the reaction vessel is then heated to facilitate the reaction. As is shown in Scheme 2, the heating radicalizes the AIBN, forming two 2-cyanopropyl 3⁰ radicles and nitrogen gas. As can be seen, these two 2-cyanopropyl 3⁰ radicles attack the carbon-sulfur bonds holding the end-group residue of the chain transfer agent to the ω-chain end of bottlebrush RAFT polymer, thereby removing it from the end of the polymer. As there will be a large excess of 2-cyanopropyl 3⁰ radicles present, the sulfur-containing compound that has been removed and replaced by a 2-cyanopropyl end capping group as shown in Scheme 2.

The temperature necessary to radicalize sufficient AIBN will depend upon the planned reaction time, but the vessel should be heated to a temperature of at least 40° C. to facilitate the reaction but should not be heated in excess of 60° C. In some embodiments, the reaction vessel is heated to a reflux temperature for the reaction mixture. The reaction vessel may be heated by any suitable method, including, but not limited to and oil bath, a water bath, or an electrical heating plate or coil, but the reaction vessel is preferably heated in an oil bath. As will be apparent to those of skill in the art, the higher the reaction temperature, the shorter the reaction time required for the reaction. In some embodiments, the reaction vessel is submerged into an oil bath and heated to 80° C., and the reaction was run for 3-4 hours. In some other embodiments, the reaction vessel is submerged into an oil bath and refluxed at 65-70° C. for 5-6 hours.

After the reaction shown in Scheme 2 has been completed, the reaction mixture may be dried in resulting optically clear bottlebrush RAFT polymer purified using any methods known in the art for that purpose. In some embodiments, the reaction mixture may be dried under reduced pressure using a rotovap (typically at 80-100 mbar, 35° C.) and the resulting viscous liquid washed repeatedly with methanol, redissolved in THF, and passed through a 1 μm PTFE filter. Finally, all volatiles are removed (at 80-100 mbar, 35° C.), and the resulting transparent and colorless viscous liquid polymer melt is further dried under high vacuum overnight at room temperature.

In one or more embodiments, the sulfur containing end-group residue of the chain transfer agent on the bottlebrush RAFT polymers of the present invention may be removed and replaced with a 2-cyanopropyl end capping group as described in Examples 12 and 13, below.

In a second aspect, the present invention is directed to artificial intraocular lenses (IOLs) for use in treating cataracts comprising a lens shell having a sealed cavity substantially filled with an optically clear filler material containing one or more of the bottlebrush polymers and copolymers described above. In these embodiments, the bottlebrush polymers and copolymers will have a refractive index (nᵣ) of from about 1.40 to about 1.48 and a complex viscosity of from about 0.4 Pa·s to about 12 Pa·s. In various embodiments, the optically clear filler material is solvent-free.

In various embodiments, the artificial lens will comprise a flexible lens shell or bag containing the filling material comprising one or more of the methacrylate-based bottlebrush polymers or copolymers discussed above. As will be understood by those skilled in the art, the lens of the eye is acted upon by the muscles of accommodation which change the shape of the lens to allow the eye to focus over a range of distances (FIGS. 2, 3). People with young healthy eyes can focus on objects at near through a process called accommodation. During accommodation, there is an increase in the optical power of the eye's crystalline lens due to an increase in lens axial thickness, an increase in curvature of the lens anterior and posterior surfaces, and a decrease in lens diameter.

An IOL according to one or more embodiment of the present invention, is shown in FIGS. 2-4. Referring first to FIG. 2, the IOL may be comprised of a thin flexible shell 2a filled with the optically clear filling medium 2b. in one or more embodiments, the thin shell may be between 20 microns and 1 mm in thickness and may be composed of a flexible silicone elastomer, hydrophobic acrylic, or other flexible and biocompatible material. In these embodiments, the filling medium is the optically clear, biocompatible, flexible bottlebrush polymer material described herein. In these embodiments, the refractive index of the filling material is selected to create an IOL with a predetermined power. An inserter device 2c is used to insert the prefilled IOL through a limbal incision 2i into the eye's capsular bag 2f. The IOL may be adaptive such that when the muscles of accommodation 2d contract, the shape of the IOL changes so the IOL provides more diopters of power, allowing the eye to focus at near. The eye's cornea 2g and iris 2h are illustrated as are the zonular fibers 2e that attach the ciliary muscles 2d to the eye's capsular bag 2f. The IOL in this embodiment is filled with the filling medium and sealed before it enters the inserter device which then inserts the lens into the eye's capsular bag. It is also contemplated that an IOL fully preformed of the polymer described herein, and not including an exterior shell, might be provided.

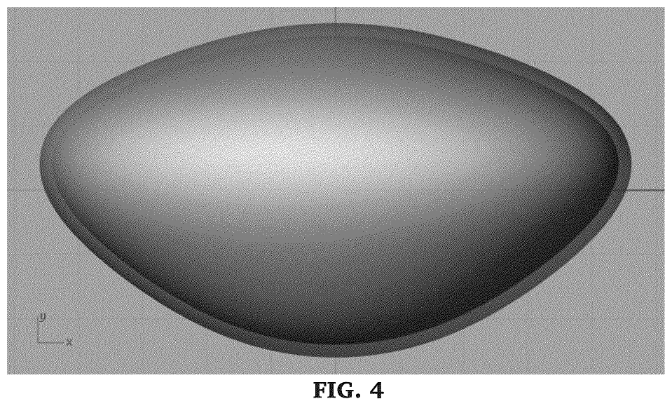

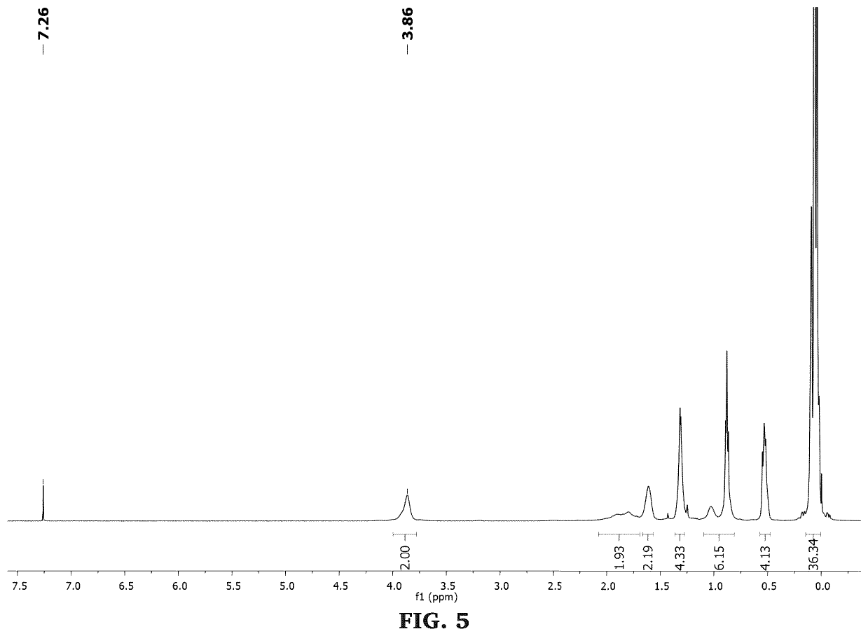

Referring now to FIG. 3, the thin flexible shell of the IOL 3a is inserted into the capsular bag 3f with an inserter device 3b. The inserter device 3b is used to inject the optically clear filling medium through a thin cannula 5c into the shell. The shell could have a one-way valve or a plug. The shell could be made of a self-sealing material. Alternatively, a sealant could be placed on the shell after insertion of the filling material. Again, the thin shell may be between 20 microns and 1 mm in thickness and is composed of a flexible silicone elastomer, hydrophobic acrylic, or other flexible and biocompatible material. The filling material is the optically clear, biocompatible, flexible bottlebrush polymer material described above and is capable of being produced in a variety of refractive indexes, as described herein. The refractive index of the filling material is selected to create an IOL with a predetermined power. In these embodiments, the IOL is adaptive such that when the muscles of accommodation contract 3d, the shape of the IOL changes so the IOL provides more diopters of power, allowing the eye to focus at near. The eye's cornea 3g, iris 3h, and vitreous 3j are illustrated as are the zonular fibers 3e that attach the ciliary muscles 3d to the eye's capsular bag 3f. The IOL in this embodiment is inserted into the capsular bag, and then filled with the filling medium and sealed inside the eye's capsular bag. FIG. 4 is an image of one version of the A-IOL in which the shell material is of uniform thickness.

According to the Helmholtz theory of accommodation, when an eye is focused at distance, the circular ciliary muscle is relaxed and the zonules pull on the lens, flattening it. When the eye focuses on a near object, the ciliary muscle contracts, and the lens zonules slacken. With the decreased zonular tension, the lens becomes thicker and more convex. This rounder lens leads to an increase in the dioptric power of the eye, allowing for near vision. In the Helmholtz theory, the zonules are relaxed during accommodation and are under tension when accommodation ends. (Glasser 2006)

As set forth above, the natural lens loses its elasticity over time, growing thicker and less flexible leading to presbyopia. With age, the lens becomes thicker and more opaque, leading to blurred vision and cataracts. The artificial lens according to the present invention is implanted in the eye of a patient to replace a lens that has become thicker, less flexible, and more opaque with age. This A-IOL should have a refractive index (nᵣ) between 1.40-1.48 and complex viscosity to allow it to be deformed by the muscles of the eye to allow the eye to focus. The present disclosure provides a solution for presbyopia and cataracts with an accommodating intraocular lens that can change shape in response to the muscles of accommodation and obviate the need for eyeglasses and contact lenses by providing clear vision over a range of distances.

In some embodiments, the artificial lens will be an accommodating intraocular lens (A-IOL). In some embodiments, the intraocular lens will not be accommodating. In some embodiments, the IOL may be an intraocular lens as described in U.S. Pat. No. 10,278,810, US Patent Application Publication 2019/0321163 A1 (Continuation), or International Application Number PCT/US20/52316, the disclosures of which are incorporated herein by reference in their entirety.

### EXAMPLES

The following examples are offered to more fully illustrate the invention but are not to be construed as limiting the scope thereof. Further, while some of examples may include conclusions about the way the invention may function, the inventors do not intend to be bound by those conclusions but put them forth only as possible explanations. Moreover, unless noted by use of past tense, presentation of an example does not imply that an experiment or procedure was, or was not, conducted, or that results were, or were not actually obtained. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

### Materials

Unless otherwise noted, solvents were received from Fisher Scientific as ACS grade and used without further purification. 4-Cyano-4-[(dodecylsulfanylthiocarbonyl) sulfanyl] pentanoic acid (Chain transfer agent #1 (CTA1), 97% HPLC, Sigma-Aldrich, CAS# 870196-80-8), 4-cyano-4-(thiobenzoylthio)pentanoic acid (CTA2, 97%, Strem Chemicals, CAS# 201611-92-9), sodium borohydride (NaBH₄, 99.99%, Sigma-Aldrich), chloroform-d (CDCl₃, 99.8 atom % D, contains 0.03% v/v TMS, Sigma-Aldrich), and methylene chloride-d2 (CD₂Cl₂, 99.8 atom % D, Acros Organics) were used as received. 2,2′-Azobis(2-methylpropionitrile) (AIBN, 98%, Sigma-Aldrich) was recrystallized from MeOH. Monomethacryloxypropyl terminated PDMS-asymmetric (PDMS-MA700, MCR-M07, MW=600-800 g/mol, Gelest), oligo(ethylene glycol) methyl ether methacrylate (OEGMA, Mₙ=500 g/mol, Sigma-Aldrich), benzyl methacrylate (BzMA, 96%, Sigma-Aldrich), ethylene glycol phenyl ether methacrylate (EGPhEMA, Sigma-Aldrich), 2,2,2-trifluoroethyl methacrylate (TFEMA, TCI Chemicals), and hydroxypropyl acrylate (HPA, 95%, Sigma-Aldrich) were freshly purified by passing through a short column of basic alumina before use.

### Instrumentation

NMR spectroscopy analysis of the samples was collected using a Bruker Advance Neo 500 MHz multinuclear NMR spectrometer. Chemical shifts are reported in ppm (δ) and referenced to the residual CHCl₃ proton resonance at 7.26 ppm in CDCl₃ or CH₂Cl₂ proton resonance at 5.32 in CD₂Cl₂. Size exclusion chromatography (SEC) was performed using an HLC-8420GPC, EcoSEC Elite Gel Permeation Chromatography (GPC) System (Tosoh Bioscience, LLC.), equipped with UV and RI detectors, TSKgel GMHHR-M mixed bed sample column (7.8 mm ID×30 cm, 5 μm). The number average molecular mass (Mₙ), weight average molecular mass (M_{w}), and molecular mass distribution (Ð_{M}) for each sample (with a concentration of 5-10 mg/mL) were calculated using a calibration curve determined from poly(styrene) standards (PStQuick C and D, Tosoh Bioscience LLC) with CHCl₃ as eluent flowing 0.5 mL/min at 40° C. Also, SEC was performed on two Agilent PLgel mixed-C columns (105 Å, 7.5×300 mm, 5 μm, part number PL1110-6500) using THF (stabilized with 100 ppm BHT) as the eluent. Molecular weights were calculated using a Wyatt Dawn EOS multi-angle light scattering (MALS) detector and Wyatt Optilab DSP Interferometric Refractometer (RI). The refractive index increment (dn/dc) values were determined by online calculation based on injections of known concentration and mass. The viscosity of the neat polymer melts was measured using a TA Instruments Discovery Hybrid Rheometer 3 (DHR 3). Each polymer melt was placed between parallel plates (25 mm diameter) using a 200 μm gap, and data was collected via an angular frequency sweep ranging from 0.1 rad/s to 500 rad/s at 10% strain at 25, 37, 45, and 50° C. Refractive index measurements were performed using a Bellingham & Stanley RFM 340 with a chiller at 25 and 37° C.

### Example 1

### RAFT Polymerization of PDMS-MA (MW=600-800 g/mol)—Poly(PDMS-MA) Bottlebrush (BB) Polymers using CTAI

In a series of experiments, Poly(PDMS-MA) Bottlebrush (BB) Polymers (MW=600-800 g/mol) were produced as shown in Scheme 1 above using RAFT Polymerization of PDMS-MA at four different monomer ([M]) to RAFT agent to initiator ([I]) molar ratios with CTA1 as the RAFT agent.

### [M]:[CTA1]:[I]=50:1:0.5

A typical reversible addition-fragmentation chain-transfer (RAFT) polymerization was conducted as follows: To a Schlenk flask equipped with a Teflon coated micro stir bar, purified PDMS-MA700 macromonomer (M, 3.0 mL, ca. 50 eq.), RAFT agent (CTA1, 33.2 mg, 0.082 mmol, 1 eq.), AIBN initiator (I, 6.76 mg, 0.041 mmol, 0.5 eq.), and toluene (1.5-2.0 mL) were added and sealed with a septum. The mixture was sparged with N₂ for 5-10 minutes. Then, the flask was placed in a pre-heated oil bath at 70° C. The polymerization was run for 12-16 hours. The polymerization was quenched by opening the flask to air and adding 10-15 mL of MeOH directly to the flask. The resulting mixture was vortexed, sonicated, and placed in an ice bath for a couple of minutes. Then, the top liquid layer was decanted, and this purification step was repeated 2 to 4 more times. The final polymer was dissolved in THF, the solution was passed through a 1 μm PTFE filter, all the volatiles were removed under reduced pressure using a rotovap (typically 85-90 mbar, 35-40 ° C.), and the resulting viscous liquid polymer was dried at high vacuum at room temperature overnight. Yellow-colored, transparent, viscous liquid polymer melt was obtained (>95% monomer conversion, 1.86 g isolated yield). M_{n.theo} ca. 30,000-40,000 g/mol, M_{n.GPC}=34,220 g/mol, M_{w.GPC}=38,600 g/mol, Ð_{M}=1.13 (dn/dc=0.041). Refractive index (n)=1.43297. ¹H NMR (400 MHZ, CDCl₃, 25° C.) δ=3.80 (b, 2H, —CO₂CH₂-), 2.10-1.72 (b, 2H, —CH₂-), 1.61 (b, 2H, —CO₂CH₂CH₂-), 1.38-1.25 (b, 4H, —SiCH₂CH₂CH₂CH₃), 1.11-0.78 (b, 6H, —CH₃), 0.60-0.44 (b, 4H, —SiCH₂-), 0.16-0.01 (b, 36H, —Si(CH₃)₂) (Sec, FIG. 5)

### [M]:[CTA]:[I]=20:1:0.5

PDMS-MA700 macromonomer (M, 1.0 mL, ca. 20 eq.), RAFT agent (CTA1, 33.2 mg. 0.082 mmol, 1 eq.), AIBN initiator (I, 6.76 mg. 0.041 mmol, 0.5 eq.), and toluene (1.0 mL) were used (>95% monomer conversion, 0.90 g isolated yield).

### [M]:[CTA]:[I]=100:1:0.5

PDMS-MA700 macromonomer (M, 6.0 mL, ca. 100 eq.), RAFT agent (CTA1, 33.2 mg, 0.0823 mmol, 1 eq.), AIBN initiator (I, 6.76 mg, 0.0411 mmol, 0.5 eq.), and toluene (3.0-4.0 mL) were used (>95% monomer conversion, ca. 4.0 g isolated yield).

### [M]:[CTA]:[I]=300:1:0.5

PDMS-MA700 macromonomer (M, 5.5 mL, ca. 300 eq.), RAFT agent (CTA1, 10.3 mg, 0.0248 mmol, 1 eq.), AIBN initiator (I, 2.1 mg, 0.0124 mmol, 0.5 eq.), and toluene (3.0 mL) were used (>95% monomer conversion, ca. 5.0 g isolated yield).

### Example 2

### RAFT Polymerization of PDMS-MA to form Poly(PDMS-MA) Bottlebrush (BB) Polymers using CTAI (Larger scale)

### [M]:[CTA]:[I]=50:1:0.5

A typical reversible addition-fragmentation chain-transfer (RAFT) polymerization was conducted as shown in Scheme 3, below.

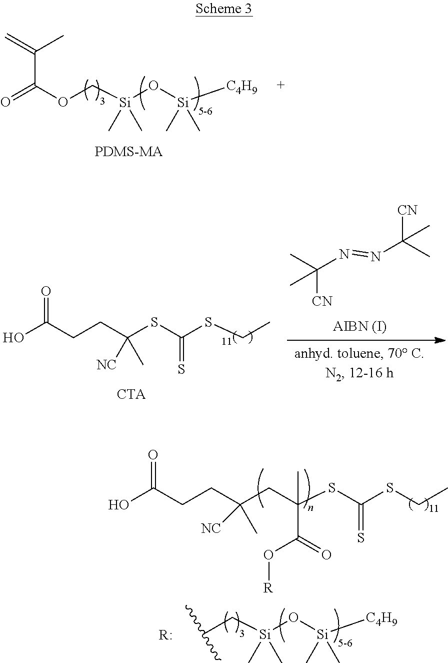

This Example is similar to Example 1 but conducted at a larger scale. To a Schlenk flask equipped with a Teflon coated micro stir bar, purified PDMS-MA macromonomer (M, 10 mL, 13.714 mmol, ca. 50 equiv), RAFT agent (CTA, 110 mg, 0.274 mmol, 1 equiv), AIBN initiator (I, 22.91 mg, 0.137 mmol, 0.5 equiv), and anhydrous toluene (5 mL) were added and sealed with a septum. The mixture was sparged with N₂ for 10-15 minutes. Then, the flask was placed in a pre-heated oil bath at 70° C. The polymerization was run for 12-16 hours (>95% % monomer conversion). The polymerization was quenched by opening the flask to air and adding methanol directly to the flask. The resulting mixture was vortexed, sonicated, and placed in an ice bath for a couple of minutes. Then, the top liquid layer was decanted, and this purification step was repeated 2 to 4 more times. The final polymer was dissolved in THF, the solution was passed through a 1 μm PTFE filter, all the volatiles were removed under reduced pressure using a rotovap (typically 90-100 mbar, 35-40° C.), and the resulting viscous liquid polymer was dried at high vacuum at room temperature overnight. Yellow colored, transparent, and viscous liquid polymer melt was obtained. ¹H NMR (400 MHZ, CDCl₃, 25° C.) δ=3.86 (b, 2H, —CO₂CH₂-), 2.10-1.72 (b, 2H, —CH₂-), 1.61 (b, 2H, -CO₂CH₂CH₂-), 1.38-1.25 (b, 4H, —SiCH₂CH₂CH₂CH₃), 1.11-0.78 (b, 6H, —CH₃), 0.60-0.44 (b, 4H, —SiCH₂-), 0.16-0.01 (b, 36H, —Si(CH₃)₂)

### Example 3

### RAFT Polymerization of PDMS-MA (MW=600-800 g/mol)—Poly(PDMS-MA) Bottlebrush (BB) Polymers using CTA2

Poly(PDMS-MA) Bottlebrush (BB) Polymers (MW=600-800 g/mol) were produced as shown in Scheme 4 below by RAFT Polymerization of PDMS-MA at with CTA2 as the RAFT agent with a macromonomer ([M]) to RAFT agent to Initiator ([I]) of 50:1:0.5.

PDMS-MA700 macromonomer ([M], 0.67 mL, ca. 50 eq.), RAFT agent ([CTA2], 5.3 mg, 0.0180 mmol, 1 eq.), initiator ([I], ca. 3 mg, 0.5 eq.) ([M]:[CTA2]:[I]=50:1:0.5), and toluene (1.0 mL) were used. The reaction was quenched, and the polymer purified as set forth in Example 1 above, to produce a pink colored, transparent, viscous liquid polymer melt after purification (>90% monomer conversion).

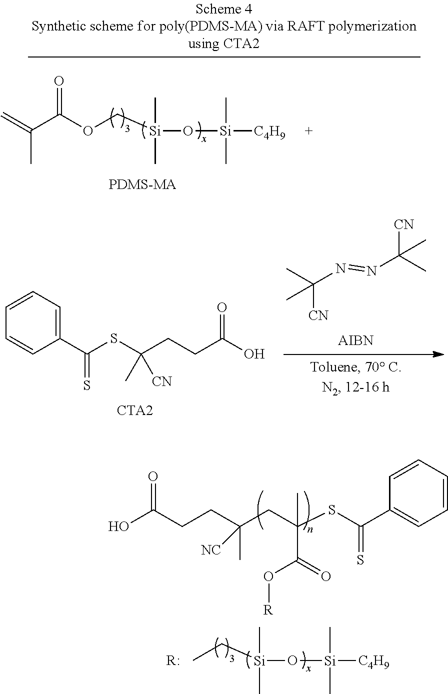

### Example 4

### RAFT Polymerization of OEGMA (Mₙ=500 g/mol)—Poly(OEGMA) Bottlebrush Polymers

Poly(OEGMA) Bottlebrush Polymers were formed by RAFT Polymerization of OEGMA (Mₙ=500 g/mol) as shown in Scheme 5 below ([M]:[CTA]:[I]=100:1:0.5).

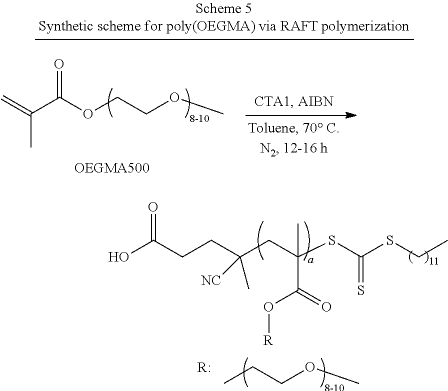

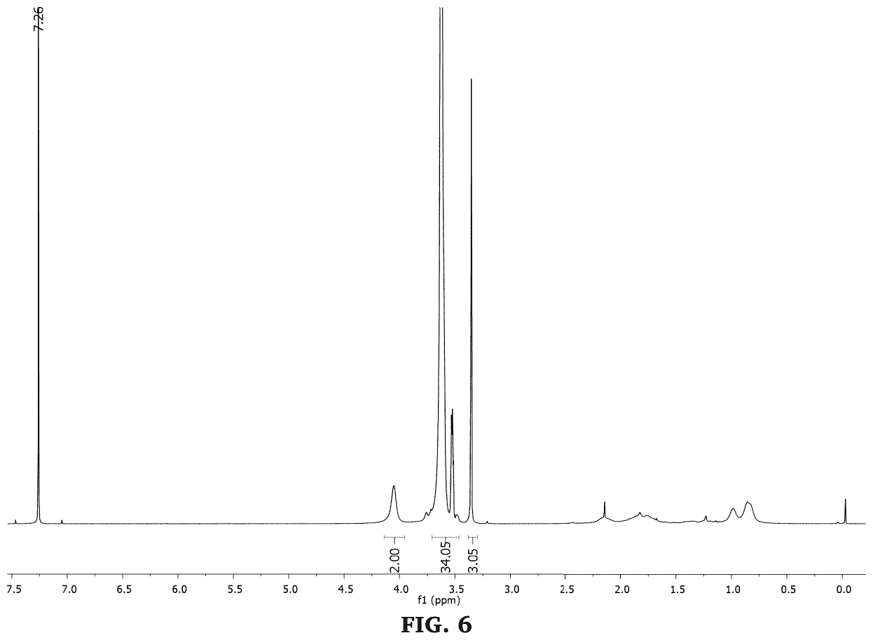

To a Schlenk flask equipped with a Teflon coated micro stir bar, purified OEGMA500 macromonomer (M, 1.13 mL, ca. 100 eq.), RAFT agent (CTA1, 9.83 mg, 0.0244 mmol, 1 eq.), AIBN initiator (I, 2-3 mg, 0.0122 mmol, 0.5 eq.), and toluene (1.0 mL) were added and sealed with a septum. The mixture was sparged with N₂ for 5-10 minutes. Then, the flask was placed in a pre-heated oil bath at 70° C. The polymerization was run for 12-16 hours. The polymerization was quenched by opening the flask to air and adding 10-15 mL of hexanes directly to the flask. The resulting mixture was vortexed, sonicated, and placed in an ice bath for a couple of minutes. Then, the top liquid layer was decanted, and this purification step was repeated 2 to 4 more times. The final polymer was dissolved in THE, the solution was passed through a 1 μm PTFE filter, all the volatiles were removed under reduced pressure using a rotovap (typically 85-90 mbar, 35-40° C.), and the resulting viscous liquid polymer was dried at high vacuum at room temperature overnight. A yellow colored, viscous liquid polymer melt was obtained (>95% monomer conversion, 1.1-1.2 g isolated yield). ¹H NMR (400 MHZ, CDCl₃, 25° C.) δ=4.05 (b, 2H, —CO₂CH₂-), 3.73-3.47 (b, 34H, —O(CH₂CH₂O)-), 3.35 (b, 3H, —OCH₃), 2.27-1.57 (b, 2H), 1.10-0.70 (b, 3H). (See, FIG. 6)

### Example 5

### RAFT Copolymerization of PDMS-MA (MW=600-800 g/mol) and OEGMA (M)=500 g/mol)—Poly(PDMS-MA-co-OEGMA) Bottlebrush Copolymers

In these experiments, Poly(PDMS-MA-co-OEGMA) Bottlebrush Copolymers were formed by RAFT Copolymerization of PDMS-MA (MW=600-800 g/mol) and OEGMA (Mₙ=500 g/mol) as shown in Scheme 6 below at various PDMS-MA:OEGMA:RAFT agent ([CTA]):initiator ([I]) ratios.

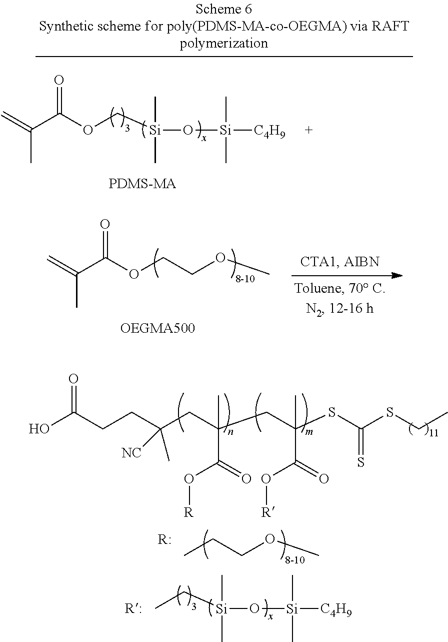

### [PDMS-MA]:[OEGMA]:[CTA]:[I]=70:30:1:0.5

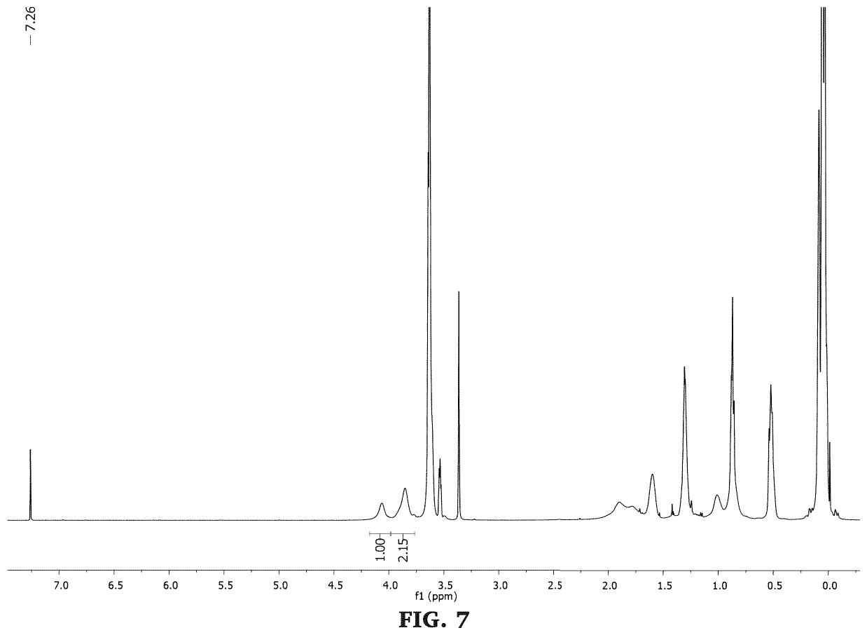

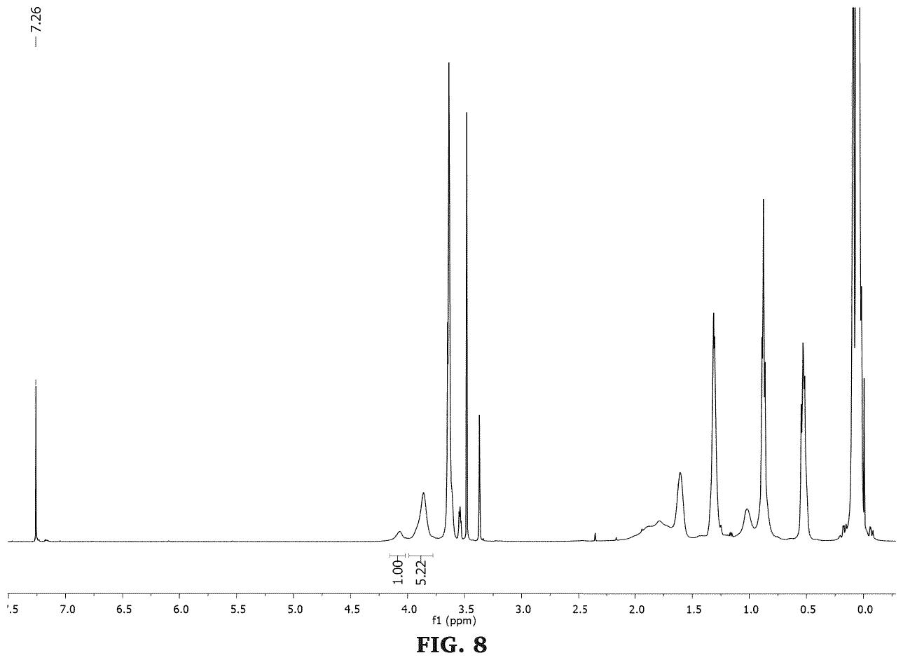

To a Schlenk flask equipped with a Teflon coated micro stir bar, purified PDMS-MA macromonomer (1.25 mL, 1.708 mmol, 70 eq.) and OEGMA500 macromonomer (0.340 mL, 0.732 mmol, 30 eq.), RAFT agent (CTA1, 9.83 mg, 0.0244 mmol, 1 eq.), AIBN initiator (I, 2-3 mg, 0.0122 mmol, 0.5 eq.), and toluene (1.0 mL) were added and sealed with a septum. The mixture was sparged with N₂ for 5-10 minutes. Then, the flask was placed in a pre-heated oil bath at 70° C. The polymerization was run for 12-16 hours. The polymerization was quenched by opening the flask to air and adding 10-15 mL of methanol directly to the flask. The resulting mixture was vortexed, sonicated, and placed in an ice bath for a couple of minutes. Then, the top liquid layer was decanted, and this purification step was repeated 2 to 4 more times. The final polymer was dissolved in THE, the solution was passed through a 1 μm PTFE filter, all the volatiles were removed under reduced pressure using a rotovap (typically 85-90 mbar, 35-40° C.), and the resulting viscous liquid polymer was dried at high vacuum at room temperature overnight. Yellow colored, viscous liquid polymer melt was obtained (>95% monomer conversion, actual compositions: 68 mol % PDMS-MA and 32 mol % OEGMA by 1H NMR spectroscopy, ca. 1.5 g isolated yield). (See, FIG. 7)

### [PDMS-MA]:[OEGMA]:[CTA]:[I]=90:10:1:0.5

To a Schlenk flask equipped with a Teflon coated micro stir bar, purified PDMS-MA macromonomer (1.60 mL, 2.196 mmol, 90 eq.) and OEGMA500 macromonomer (0.110 mL, 0.244 mmol, 10 eq.), RAFT agent (CTA1, 9.83 mg, 0.0244 mmol, 1 eq.), AIBN initiator (I, 2-3 mg, 0.0122 mmol, 0.5 eq.), and toluene (1.0 mL) were used. Methanol was used to precipitate the resulting polymer. Yellow colored, viscous liquid polymer melt was obtained after purification (>95% monomer conversion, actual compositions: 84 mol % PDMS-MA and 16 mol % OEGMA by ¹H NMR spectroscopy, ca. 1.5 g isolated yield). (See, FIG. 8)

### [PDMS-MA]:[OEGMA]:[CTA]:[I]=10:90:1: 0.5

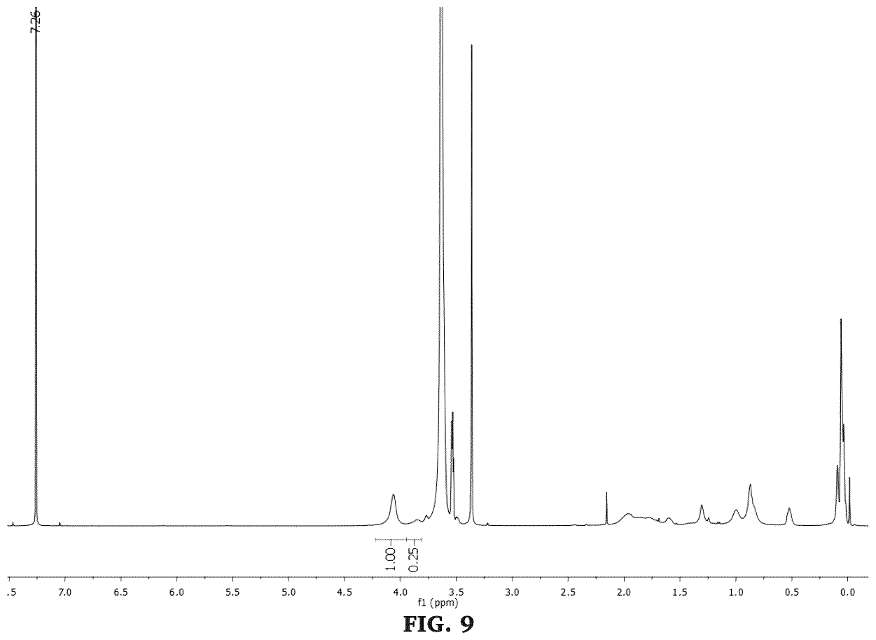

To a Schlenk flask equipped with a Teflon coated micro stir bar, purified PDMS-MA macromonomer (0.178 mL, 0.244 mmol, 10 eq.) and OEGMA500 macromonomer (1.02 mL, 2.196 mmol, 90 eq.), RAFT agent (CTA1, 9.83 mg, 0.0244 mmol, 1 eq.), AIBN initiator (I, 2-3 mg, 0.0122 mmol, 0.5 eq.), and toluene (0.5 mL) were used. Hexanes was used to precipitate the resulting polymer. Yellow colored, viscous liquid polymer melt was obtained after purification (>95% monomer conversion, actual compositions: 20 mol % PDMS-MA and 80 mol % OEGMA by ¹H NMR spectroscopy, ca. 1.1 g isolated yield). (See, FIG. 9)

### Example 6

### RAFT Copolymerization of PDMS-MA (MW=600-800 g/mol) and BzMA—Poly(PDMS-MA-co-BzMA) Comb Copolymers

In several experiments, Poly(PDMS-MA-co-BzMA) Comb Copolymers were formed by RAFT Copolymerization of PDMS-MA (MW=600-800 g/mol) and BzMA as shown in Scheme 7 below at two different PDMS-MA:BzMA:RAFT agent ([CTA]):initiator ([I]) ratios.

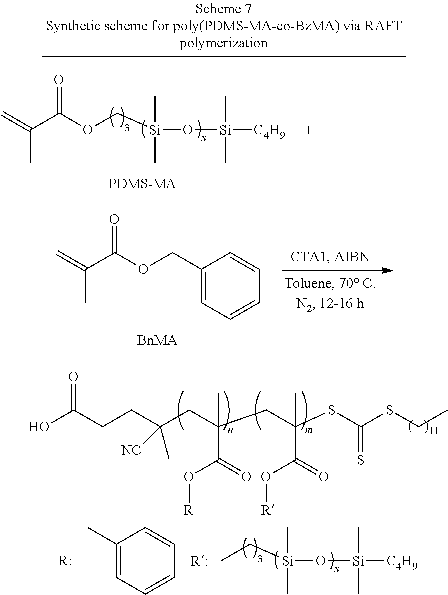

### [PDMS-MA]:[BzMA]:[CTA]:[I]=70:30:1:0.5

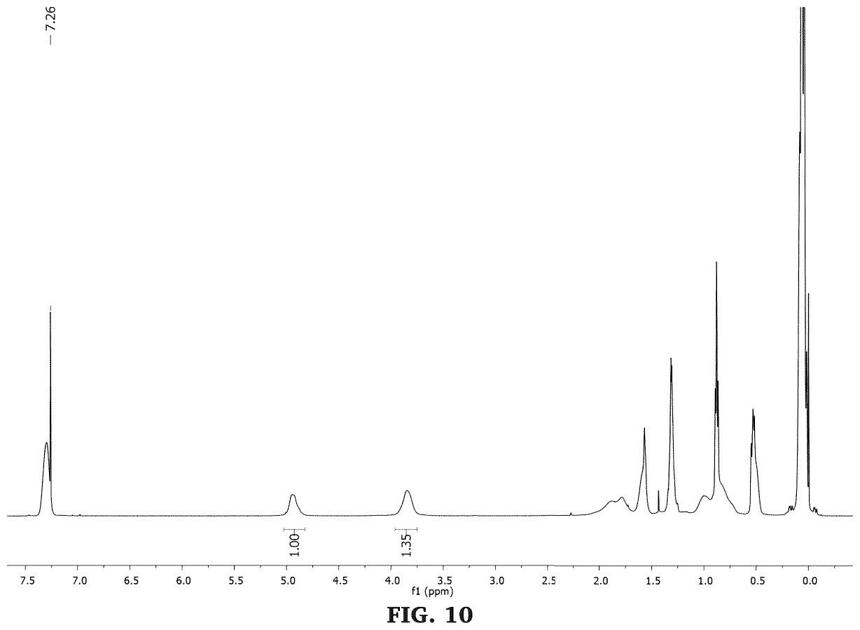

To a Schlenk flask equipped with a Teflon coated micro stir bar, purified PDMS-MA macromonomer (1.03 mL, 1.38 mmol, 70 eq.) and BzMA (0.10 mL, 0.59 mmol, 30 eq.), RAFT agent (CTA1, 6.20 mg, 0.0153 mmol, 1 eq.), AIBN initiator (I, 1-2 mg, 0.5 eq.), and toluene (1.0 mL) were used. The reaction was quenched, and the copolymer purified as set forth in Example 1 above. A yellow colored, viscous liquid polymer melt was obtained after purification (>95% monomer conversion, actual compositions: 60 mol % PDMS-MA and 40 mol % BzMA by 1H NMR spectroscopy, ca. 0.90 g isolated yield). (See, FIG. 10)

### [PDMS-MA]:[BzMA]:[CTA]:[I]=90:10:1:0.5

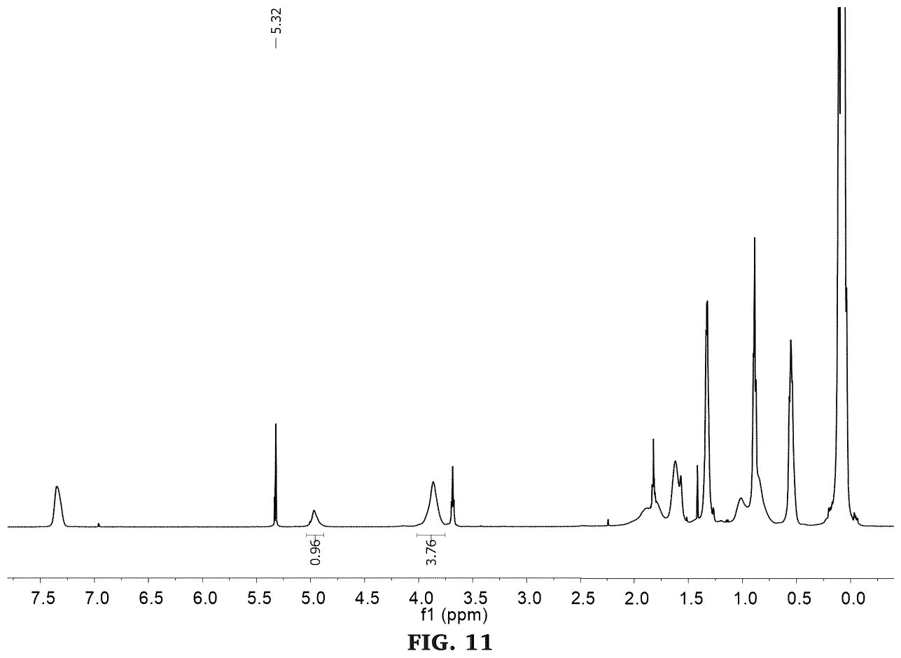

To a Schlenk flask equipped with a Teflon coated micro stir bar, purified PDMS-MA macromonomer (2.00 mL, 2.654 mmol, 90 eq.) and BzMA (0.05 mL, 0.295 mmol, 10 eq.), RAFT agent (CTA1, 11.91 mg, 0.0295 mmol, 1 eq.), AIBN initiator (I, 2-3 mg, 0.5 eq.), and toluene (1.0 mL) were used. The reaction was quenched, and the copolymer purified as set forth in Example 1 above. A yellow colored, viscous liquid polymer melt was obtained after purification (>95% monomer conversion, actual compositions: 80 mol % PDMS-MA and 20 mol % BzMA by 1H NMR spectroscopy, ca. 1.94 g isolated yield). (See, FIG. 11)

### Example 7

### RAFT Copolymerization of PDMS-MA (MW=600-800 g/mol) and EGPhEMA—Poly(PDMS-MA-co-EGPhEMA) Comb Copolymers

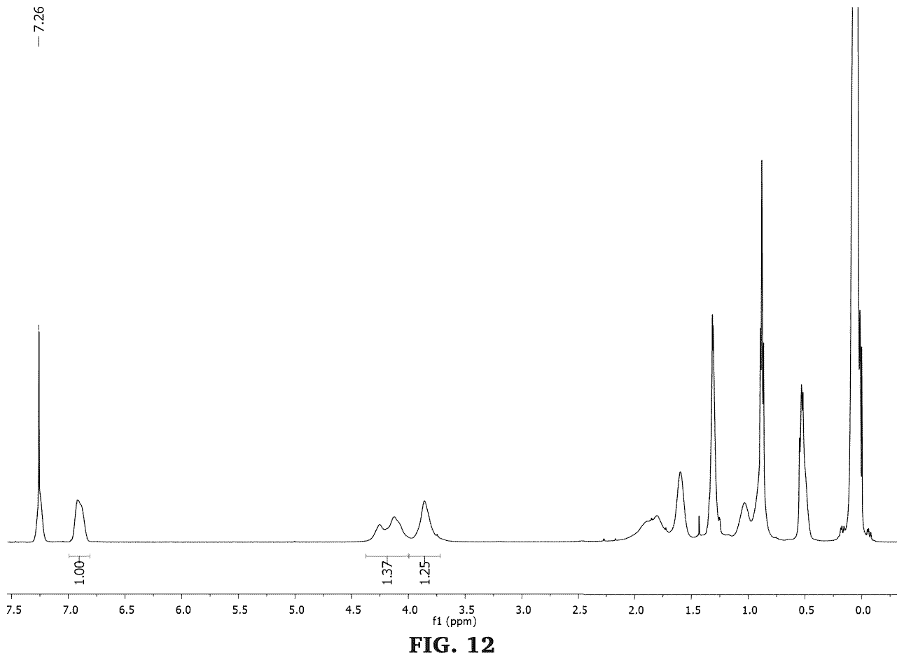

In these experiments, poly(PDMS-MA-co-EGPhEMA) comb copolymers were formed by the RAFT copolymerization of PDMS-MA (MW=600-800 g/mol) and EGPhEMA at a PDMS-MA to EGPhEMA to Raft agent ([CTA]) to initator ([I]) molar ratio of 70:30:1:0.5 ([PDMS-MA]:[EGPhEMA]:[CTA]:[I]=70:30:1:0.5). (See, FIG. 12)

To a Schlenk flask equipped with a Teflon coated micro stir bar, purified PDMS-MA macromonomer (1.214 mL, 1.734 mmol, 70 eq.) and EGPhEMA (0.142 mL, 0.743 mmol, 30 eq.), RAFT agent (CTA1, 10 mg, 0.0248 mmol, 1 eq.), AIBN initiator (I, 2-3 mg, 0.5 eq.), and toluene (1.0 mL) were used. The reaction was quenched, and the copolymer purified as set forth in Example 1 above. A yellow colored, viscous liquid polymer melt was obtained after purification (>95% monomer conversion, actual compositions: 63 mol % PDMS-MA and 37 mol % EGPhEMA by ¹H NMR spectroscopy, ca. 1.08 g isolated yield).

### Example 8

### RAFT Copolymerization of OEGMA (Mₙ=500 g/mol) and EGPhEMA—Poly(OEGMA-co-EGPhEMA) Comb Copolymers

In these experiments, Poly(OEGMA-co-EGPhEMA) Comb Copolymers were formed by RAFT Copolymerization of OEGMA (Mₙ=500 g/mol) and EGPhEMA as shown in Scheme 8, below at a OEGMA to EGPhEMA to Raft agent ([CTA]) to initator ([I]) molar ratio of 90:10:1:0.5 ([OEGMA]:[EGPhEMA]:[CTA]:[I]=90:10:1:0.5).

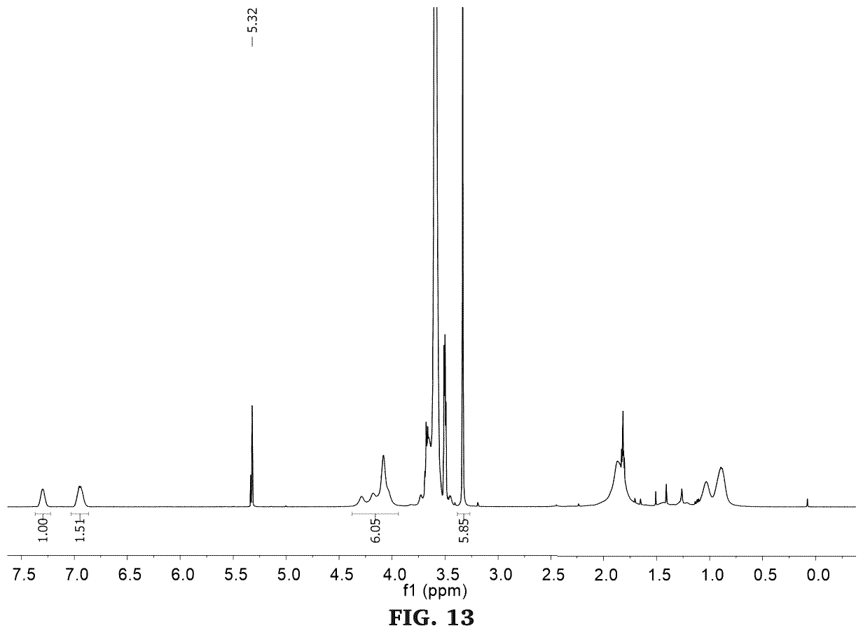

To a Schlenk flask equipped with a Teflon coated micro stir bar, purified OEGMA500 macromonomer (2.18 mL, 2.356 mmol, 90 eq.) and EGPhEMA (0.10 mL, 0.262 mmol, 10 eq.), RAFT agent (CTA1, 10.58 mg, 0.0262 mmol, 1 eq.), AIBN initiator (I, 2-3 mg, 0.0122 mmol, 0.5 eq.), and toluene (1.0 mL) were used. Hexanes was used to precipitate the resulting polymer. A yellow colored, viscous liquid polymer melt was obtained after purification (>95% monomer conversion, actual compositions: 81.5 mol % OEGMA and 18.5 mol % EGPhEMA by 1H NMR spectroscopy, ca. 1.49 g isolated yield). (See, FIG. 13)

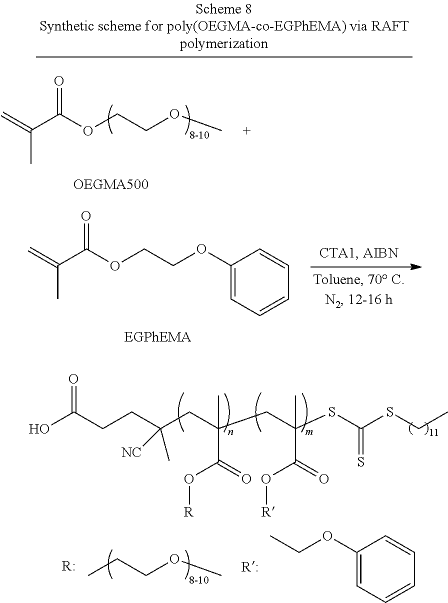

### Example 9

### RAFT Copolymerization of PDMS-MA (MW=600-800 g/mol) and TFEMA—Poly(PDMS-MA-co-TFEMA) Comb Copolymers

In these experiments, Poly(PDMS-MA-co-TFEMA) Comb Copolymers were formed by RAFT Copolymerization of PDMS-MA (MW=600-800 g/mol) and TFEMA were formed as shown in Scheme 9 below at two different PDMS-MA to TFEMA to RAFT agent to initiator molar ratios.

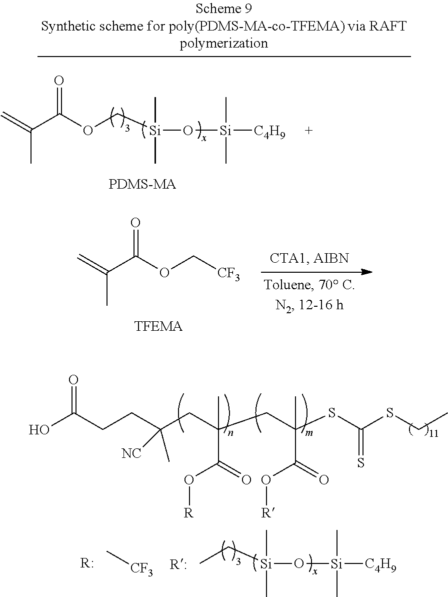

### [PDMS-MA]:[TFEMA]:[CTA]:[I]=70:30:1:0.5

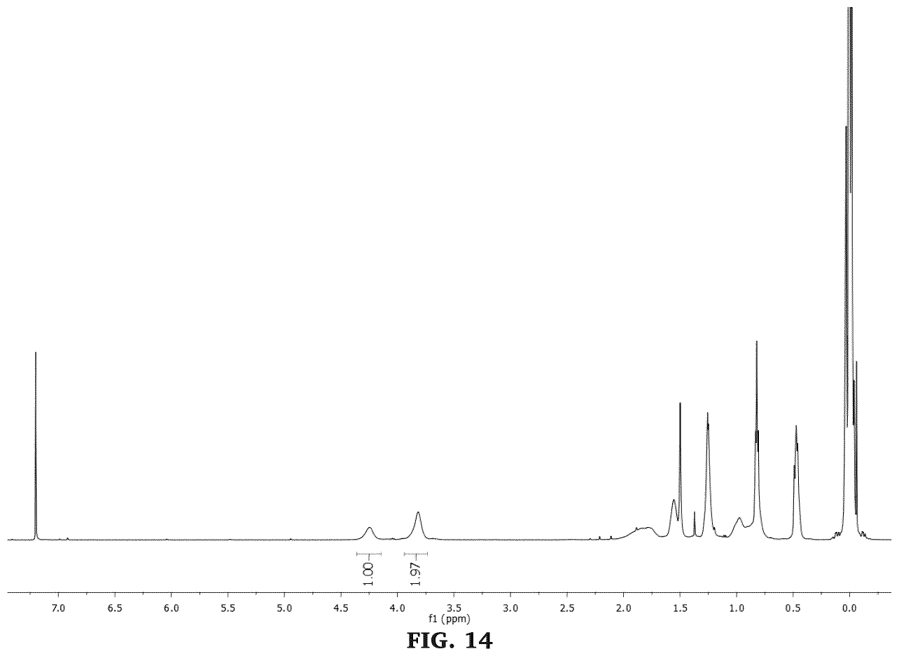

To a Schlenk flask equipped with a Teflon coated micro stir bar, purified PDMS-MA macromonomer (1.20 mL, 1.639 mmol, 70 eq.) and TFEMA (0.10 mL, 0.703 mmol, 30 eq.), RAFT agent (CTA1, 9.45 mg, 0.0234 mmol, 1 eq.), AIBN initiator (I, 2-3 mg, 0.0122 mmol, 0.5 eq.), and toluene (1.0 mL) were used. The reaction was quenched, and the copolymer purified as set forth in Example 1 above. A yellow colored, viscous liquid polymer melt was obtained after purification (>95% monomer conversion, actual compositions: 66 mol % PDMS-MA and 34 mol % TFEMA by ¹H NMR spectroscopy, ca. 1.10 g isolated yield). (See, FIG. 14)

### [PDMS-MA]:[TFEMA]:[CTA]:[I]=50:50:1:0.5

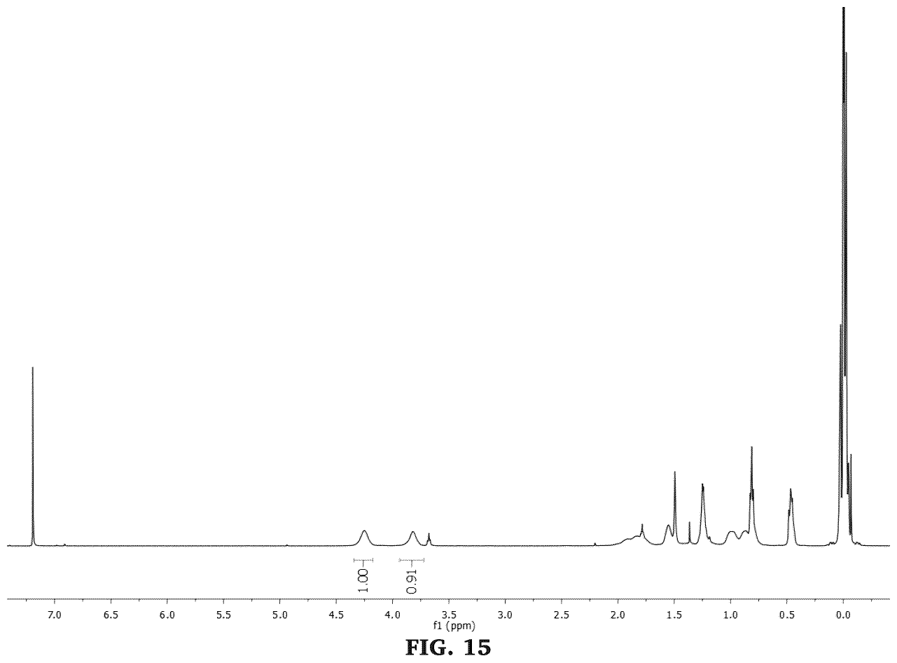

To a Schlenk flask equipped with a Teflon coated micro stir bar, purified PDMS-MA macromonomer (1.0 mL, 1.371 mmol, 50 eq.) and TFEMA (0.20 mL, 1.371 mmol, 50 eq.), RAFT agent (CTA1, 11.07 mg, 0.0274 mmol, 1 eq.), AIBN initiator (I, 2-3 mg, 0.0122 mmol, 0.5 eq.), and toluene (1.0 mL) were used. The reaction was quenched, and the copolymer purified as set forth in Example 1 above. A yellow colored, viscous liquid polymer melt was obtained after purification (>95% monomer conversion, actual compositions: 48 mol % PDMS-MA and 52 mol % TFEMA by ¹H NMR spectroscopy, ca. 0.82 g isolated yield). (See, FIG. 15)

### Example 10

### RAFT Copolymerization of PDMS-MA (MW=600-800 g/mol) and BzTAzMA—Poly(PDMS-MA-co-BzTAzMA) Comb Copolymers

In several experiments, Poly(PDMS-MA-co-BzTAzMA) Comb Copolymers were formed by RAFT Copolymerization of PDMS-MA (MW=600-800 g/mol) and BzTAzMA as shown in Scheme 10 below at a PDMS-MA:BzTAzMA:RAFT agent ([CTA]):initiator ([I]) ratio of 90:10:1:0.5.

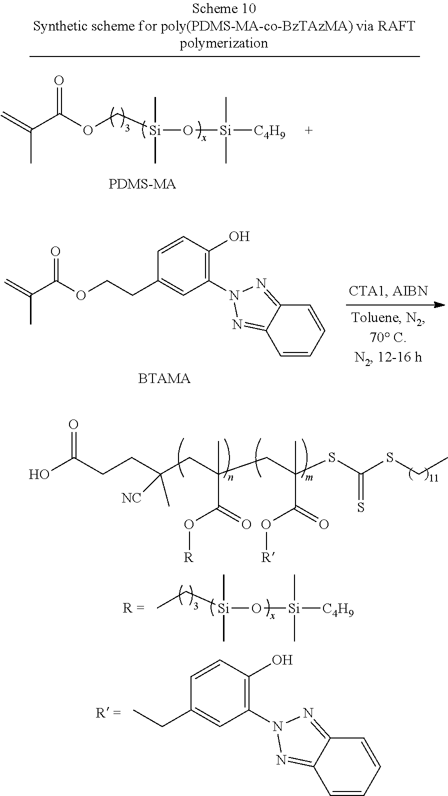

### [PDMS-MA]: [BzTAzMA]:[CTA]:[I]=90:10:1:0.5

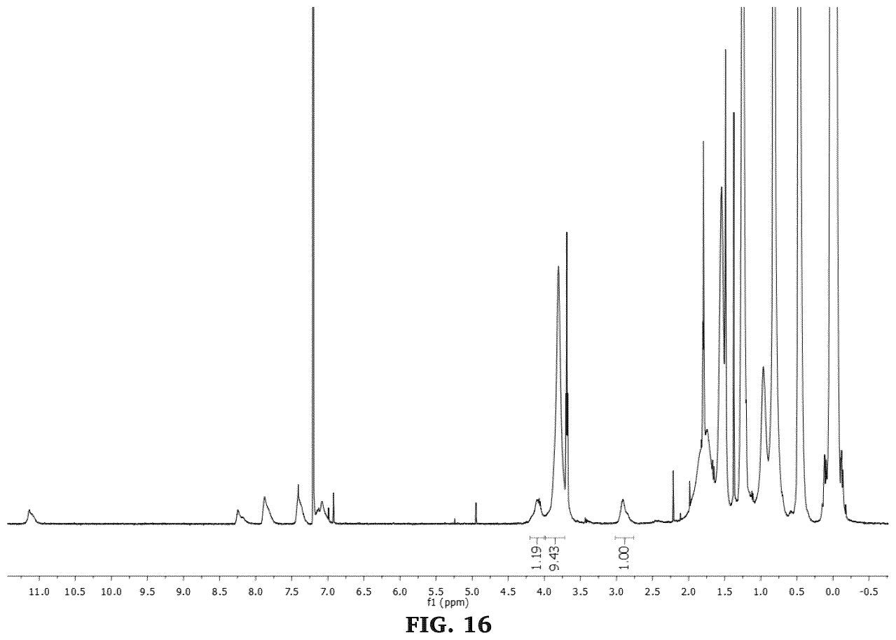

To a Schlenk flask equipped with a Teflon coated micro stir bar, purified PDMS-MA macromonomer (1.0 mL, 1.37 mmol, 90 eq.) and BzTAzMA (50 mg, 0.152 mmol, 30 eq.), RAFT agent (CTA1, 6.15 mg, 0.0153 mmol, 1 eq.), AIBN initiator (I, 1-2 mg, 0.5 eq.), and toluene (1.5 mL) were used. The reaction was quenched, and the resulting copolymer purified as set forth in Example 1 above. A yellow-colored, viscous liquid polymer melt was obtained after purification (>95% monomer conversion, ca. 0.90 g isolated yield). Mₙ ca. 60k, Ð=1.28. nᵣ=1.44041. Complex viscosity=2.93 Pa.s at 25° C., 2.02 Pa.s at 37° C. (See FIG. 16)

### Example 11

### RAFT Copolymerization of PDMS-MA (MW=600-800 g/mol) and HDFDMA—Poly(PDMS-MA-co-HDFDMA) Bottlebrush Copolymers

In these experiments, Poly(PDMS-MA-co-HDFDMA) bottlebrush Copolymers were formed by RAFT Copolymerization of PDMS-MA (MW=600-800 g/mol) and HDFDMA as shown in Scheme 11 below at two different PDMS-MA to HDFDMA to RAFT agent to initiator molar ratios.

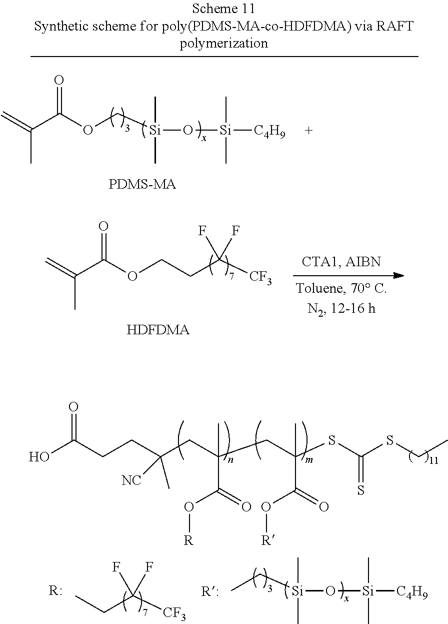

### [PDMS-MA]:[HDFDMA]:[CTA]:[I]=70:30:1:0.5

To a Schlenk flask equipped with a Teflon coated micro stir bar, purified PDMS-MA macromonomer (1.00 mL, 1.372 mmol, 70 eq.) and HDFDMA (0.196 mL, 0.588 mmol, 30 eq.), RAFT agent (CTA1, 7.91 mg, 0.0196 mmol, 1 eq.), AIBN initiator (I, 1-2 mg, 0.0098 mmol, 0.5 eq.), and toluene (1.0 mL) were used. The reaction was quenched, and the copolymer purified as set forth in Example 1 above. A yellow-colored, viscous liquid polymer melt was obtained after purification (>95% monomer conversion, actual compositions: 66 mol % PDMS-MA and 34 mol % HDFDMA by ¹H NMR spectroscopy, ca. 0.90 g isolated yield).

### [PDMS-MA]:[HDFDMA]:[CTA]:[I]=80:20:1:0.5

To a Schlenk flask equipped with a Teflon coated micro stir bar, purified PDMS-MA macromonomer (1.00 mL, 1.372 mmol, 80 eq.) and HDFDMA (0.114 mL, 0.343 mmol, 30 eq.), RAFT agent (CTA1, 6.92 mg, 0.0172 mmol, 1 eq.), AIBN initiator (I, 2-3 mg, 0.0122 mmol, 0.5 eq.), and toluene (1.0 mL) were used. The reaction was quenched, and the copolymer purified as set forth in Example 1 above. A yellow-colored, viscous liquid polymer melt was obtained after purification (>95% monomer conversion, actual compositions: 76 mol % PDMS-MA and 24 mol % HDFDMA by ¹H NMR spectroscopy, ca. 0.80 g isolated yield).

### Example 12]

### End-Group Removal (EGR) of RAFT Bottlebrush (BB) Polymers via AIBN Treatment

In these experiments, end-groups were removed from RAFT bottlebrush polymers using AIBN as shown in Scheme 12, below.

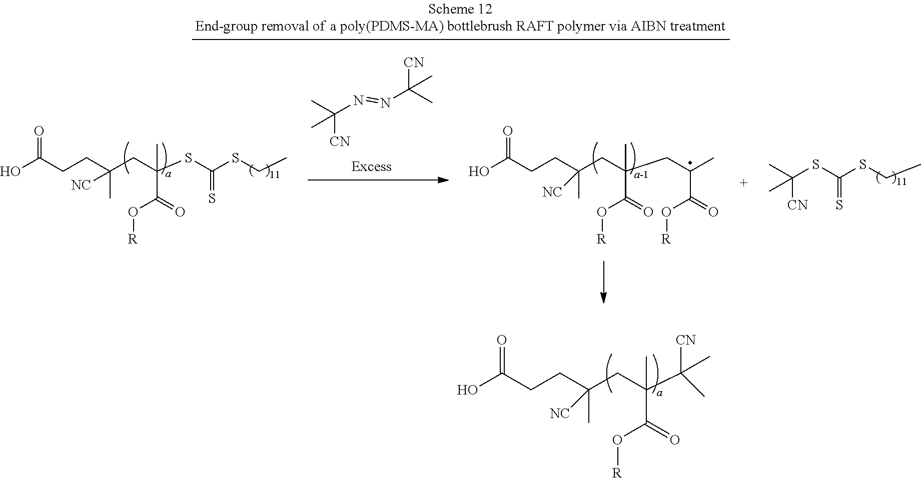

First, polymer was dissolved in toluene (ca. 100-200 mg/mL solution), and AIBN was added into the flask (20 eq. relative to RAFT agent used to synthesize the polymer). The flask was equipped with a Teflon coated stir bar and sealed with a rubber septum. The resulting mixture was sparged with N₂ for 20-30 min (>1 ml/min). Then, the flask was submerged into an oil bath and heated to 80° C., and the reaction was run for 3-4 hours (Half-life, t_{1/2}, of AIBN at 80° C. is around 90 min). After the reaction, the flask was allowed to cool back to room temperature and another 20 eq. of AIBN was added to the flask. The mixture was sparged with N₂ and heated at 80° C. for another 3-4 hours. After a total of three AIBN treatments, the reaction mixture was dried under reduced pressure using a rotovap (typically at 80 mbar, 45° C.). The viscous liquid was washed with methanol five times, redissolved in THF, and passed through a 1 μm PTFE filter. Finally, all volatiles were removed (at 90-100 mbar, 35° C.), and the resulting viscous liquid polymer melt was further dried under high vacuum overnight at room temperature.

### Example 13

### End-Group Removal of the Chain Transfer Agent at the ω-Chain End of Poly(PDMS-MA) Bottlebrush RAFT Polymer via AIBN Treatment

Chain-transfer agent (CTA) end-groups were removed from polymers using excess AIBN as shown in Scheme 2 above. First, polymer was dissolved in THF (ca. 0.1 g/mL solution), and AIBN was added into the flask (20 equiv. relative to CTA used to synthesize the polymer). The flask was equipped with a Teflon coated stir bar and sealed with a rubber septum. The resulting mixture was sparged with N₂ for 20-30 min (>1 mL/min). Then, the flask was submerged into an oil bath and refluxed at 65-70° C. for 5-6 hours (Half-life, tin, of AIBN at 70° C. is around 5 hours). After the AIBN treatment, the reaction mixture was dried under reduced pressure using a rotovap (typically at 80-100 mbar, 35° C.). The viscous liquid was washed with methanol five times, redissolved in THF, and passed through a 1 μm PTFE filter. Finally, all volatiles were removed (at 80-100 mbar, 35° C.), and the resulting transparent and colorless viscous liquid polymer melt was further dried under high vacuum overnight at room temperature. (8.3 g overall isolated yield; Refractive index (nᵣ)=1.429; Viscosity=1.05 Pa.s at 25° C., 0.87 Pa.s at 37° C.).

### Example 14

### THF SEC Analysis of Poly(PDMS-MA)

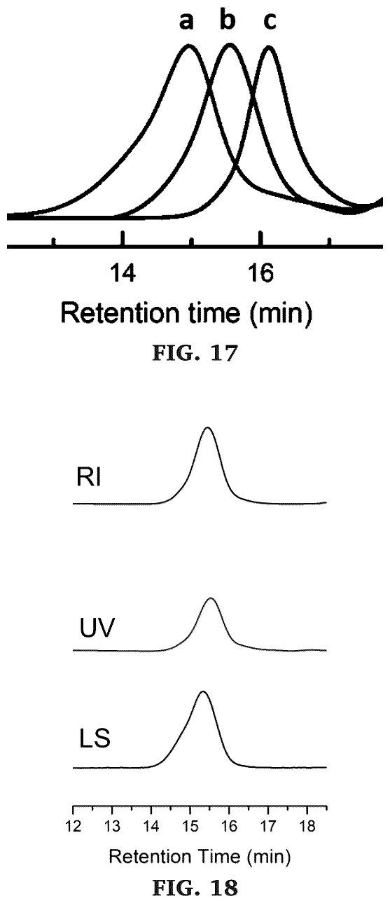

Poly(PDMS-MA) polymer of different molecular weights were analyzed using THF SEC. In a first set of experiments, SEC was performed on three poly(PDMS-MA) polymers using two Agilent PLgel mixed-C columns (105 Å, 7.5×300 mm, 5 μm, part number PL1110-6500) using THF (stabilized with 100 ppm BHT) as the eluent and the molecular weights were calculated using a Wyatt Dawn EOS multi-angle light scattering (MALS) detector and Wyatt Optilab DSP Interferometric Refractometer (RI). The resulting THF SEC traces of poly(PDMS-MA) with three different molecular weights. (a: Mₙ=13,370 g/mol, b: Mₙ=27,160 g/mol, c: Mₙ=67,900 g/mol) are shown in FIG. 17.

In a second set of experiments, a poly(PDMS-MA) polymer samples were analyzed by SEC using an HLC-8420GPC, EcoSEC Elite Gel Permeation Chromatography (GPC) System (Tosoh Bioscience, LLC.), equipped with UV and RI detectors, TSKgel GMHHR-M mixed bed sample column (7.8 mm ID×30 cm, 5 μm). The number average molecular mass (Mₙ), weight average molecular mass (M_{w}), and molecular mass distribution (PM) for each sample (with a concentration of 5-10 mg/mL) were calculated using a calibration curve determined from poly(styrene) standards (PStQuick C and D, Tosoh Bioscience LLC) with CHCl₃ as eluent flowing 0.5 mL/min at 40° C. FIG. 18 shows THF SEC traces of poly(PDMS-MA) with three detectors. top trace: refractive index detector, middle trace: UV detector at 254 nm, bottom trace: light scattering detector.

### Example 15

### Complex Viscosity/RI Analysis

The viscosity of neat polymer melts was measured using a TA Instruments Discovery Hybrid Rheometer 3 (DHR 3). Each polymer melt was placed between parallel plates (25 mm diameter) using a 200 μm gap, and data was collected via an angular frequency sweep ranging from 0.1 rad/s to 500 rad/s at 10% strain at 25, 37, 45, and 50° C. Refractive index measurements were performed using a Bellingham & Stanley RFM 340 with a chiller at 25 and 37° C.

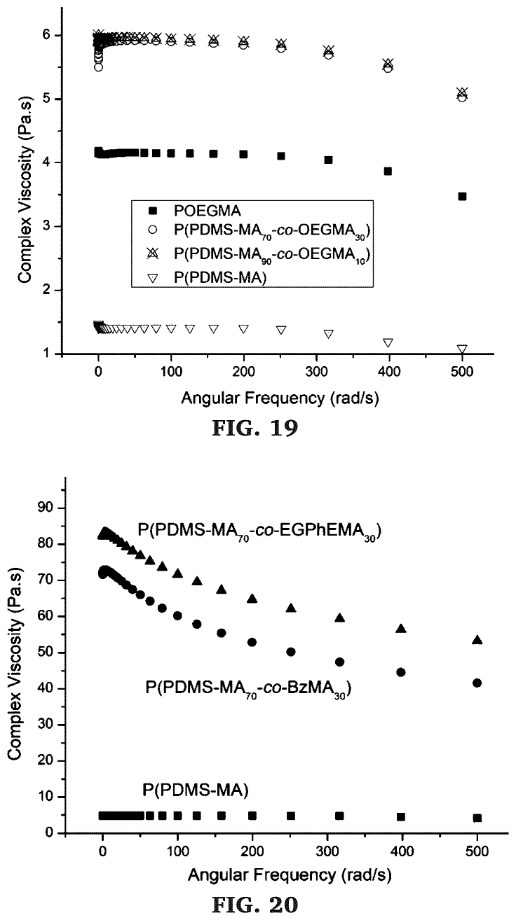

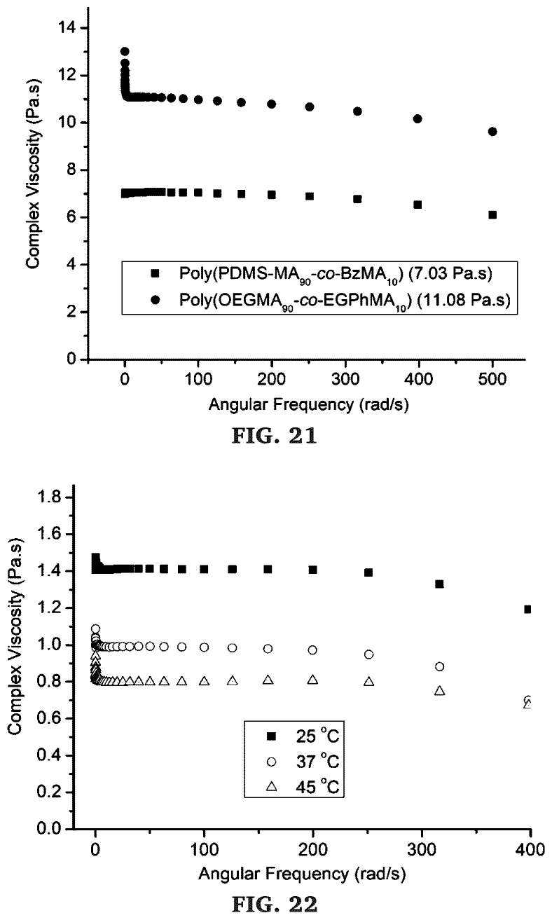

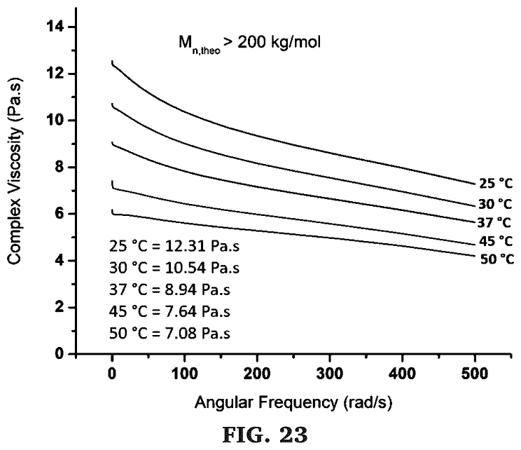

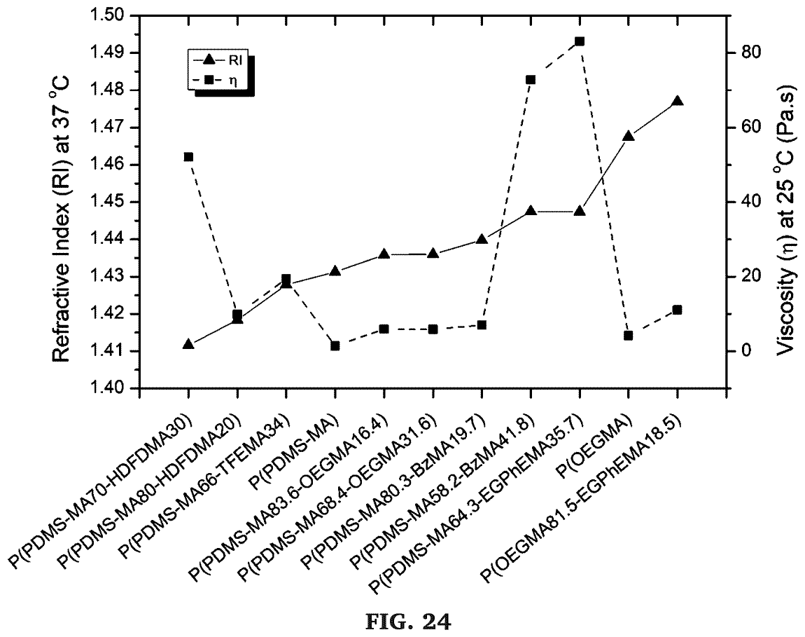

The complex viscosity measurements of poly(PDMS-MA), POEGMA, and their random copolymers with various ratios at 25° C. are shown in FIG. 19. The complex viscosity measurements of poly(PDMS-MA), poly(PDMS-MA₇₀-co-BzMA₃₀), and poly(PDMS-MA₇₀-co-EGPhEMA₃₀) at 25° C. are shown in FIG. 20. The complex viscosity measurements for poly(PDMS-MA₉₀-co-BzMA₁₀) (RI=1.43981 at 37° C.) and poly(OEGMA₉₀-co-EGPhEMA₁₀) (RI=1.47694 at 37° C.) at 25° C. are shown in FIG. 21. The complex viscosity measurements for poly(PDMS-MA) at various temperatures (M_{n.theo}=30,000-40,000 g/mol) are shown in FIG. 22. As expected, viscosity drops proportionally as the temperature is increased. The complex viscosity measurements of poly(PDMS-MA) at various temperatures (M_{n.theo}>200,000 g/mol) are shown in FIG. 23. Finally, FIG. 24 is a graph comparing refractive index (RI) and viscosity for polydimethylsiloxane methacrylate (PDMS-MA), heptadecafluorodecyl methacrylate (HDFDMA), trifluoroethyl methacrylate (TFEMA), oligoethyleneglycol methacrylate (OEGMA), Benzyl methacrylate (BzMA), and ethylene glycol phenyl ether methacrylate (EGPhEMA).

### REFERENCES

Hao X, Jeffery J L, Wilkie J S, Meijs G F, Clayton A B, Watling J D, Ho A, Fernandez V, Acosta C. Yamamoto H, Aly MGM, Parel J-M, Hughes TC (2010) Functionalised polysiloxanes as injectable, in situ curable accommodating intraocular lenses. Biomaterials, 31(32):8153-8163. https://doi.org/10.1016/j.biomaterials.2010.07.065
Koopmans S A, Terwee T, Barkhof J, Haitjema H J, Kooijman A C (2003) Polymer refilling of presbyopic human lenses in vitro restores the ability to undergo accommodative changes. Investigative Ophthalmology & Visual Science, 44(1):250-257. https://doi.org/10.1167/iovs.02-0256
Koopmans S A, Terwee T, Glasser A, Wendt M, Vilupuru A S, Vilipuru A S, Kooten T G van, Norrby S, Haitjema H J, Kooijman A C (2006) Accommodative lens refilling in rhesus monkeys. Investigative Ophthalmology & Visual Science, 47(7): 2976-2984. https://doi.org/10.1167/iovs.05-1346
Koopmans S A, Terwee T, Hanssen A, Martin H, Langner S, Stachs O, Kooten T G van (2014) Prevention of capsule opacification after accommodating lens refilling: Pilot study of strategies evaluated in a monkey model. Journal of Cataract & Refractive Surgery, 40(9):1521-1535. https://doi.org/10.1016/j.jcrs.2014.02.034
Hao X, Jeffery J L, Le T P T, McFarland G, Johnson G, Mulder R J, Garrett Q, Manns F, Nankivil D, Arrieta E, Ho A, Parel J-M, Hughes T C (2012) High refractive index polysiloxane as injectable, in situ curable accommodating intraocular lens. Biomaterials, 33(23):5659-5671. https://doi.org/10.1016/j.biomaterials.2012.04.052
Nishi O, Nakai Y, Mizumoto Y, Yamada Y (1997) Capsule opacification after refilling the capsule with an inflatable endocapsular balloon. Journal of Cataract and Refractive Surgery, 23(10): 1548-1555. https://doi.org/10.1016/s0886-3350(97)80028-4
Young M, Gamat G (2016) Clinical trial of new accommodating IOL reveals promising results. Eyeworld. https://www.eyeworld.org/article-clinical-trial-of-new-accommoda ting-iol-reveals-
Glasser, Adrian. “Accommodation: Mechanism and Measurement,” Ophthalmol Clin N Am, 19 (2006), pp 1-12).

In light of the foregoing, it should be appreciated that the present invention significantly advances the art by providing optically clear bottlebrush polymers and copolymers that are structurally and functionally improved in a number of ways and that have tunable viscosity and optical properties for use in intraocular lenses. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

1. A bottlebrush polymer comprising a homopolymer or copolymer of a methacrylate macromolecule monomer selected from the group consisting of monomethacryloxypropyl terminated polydimethylsiloxane, asymmetric (PDMS-MA) and oligo(ethylene glycol) methacrylate (OEGMA) or a copolymer of at least one of PDMS-MA and OEGMA and at least one methacrylate or acrylate monomer selected from the group consisting of 2,2,2-trifluoroethyl methacrylate (TFEMA), 3,3,4,4,5,5,6,6,7,7,8,8,9,9,10,10,10-heptadecafluorodecyl methacrylate (HDFDMA), benzyl methacrylate (BzMA), 2-[3-(2H-Benzotriazol-2-yl)-4-hydroxyphenyl]ethyl methacrylate (BzTAzMA), ethyleneglycol phenylether methacrylate (EGPhEMA), hydroxyethyl methacrylate (HEMA), 2,2,2-trifluoroethyl acrylate (TFEA), 3,3,4,4,5,5,6,6,7,7,8,8,9,9,10,10,10-heptadecafluorodecyl acrylate (HDFDA), benzyl acrylate (BzA), 2-[3-(2H-Benzotriazol-2-yl)-4-hydroxyphenyl]ethyl acrylate (BzTAzA), ethyleneglycol phenylether acrylate (EGPhEA), hydroxyethyl acrylate (HEA), and combinations thereof, and having one or more end groups derived from a reversible addition fragmentation chain-transfer (RAFT) agent, wherein said bottlebrush polymer has a complex viscosity of from about 0.5 to about 30 Pa·s at 37° C.

2. The bottlebrush polymer of claim 1 wherein said homopolymer or copolymer comprises the residue of an ultra-violet (UV) light blocking methacrylate monomer.

3. (canceled)

4. The bottlebrush polymer of claim 1 wherein said bottlebrush polymer is a copolymer of monomethacryloxypropyl terminated polydimethylsiloxane, asymmetric (PDMS-MA) and oligo(ethylene glycol) methacrylate (OEGMA) formed by RAFT polymerization and comprising from about 10 to about 95 mole percent, PDMS-MA.

5. (canceled)

6. The bottlebrush polymer of claim 1 having a refractive index of from about 1.39 to about 1.48 at 37° C.

7. (canceled)

8. (canceled)

9. The bottlebrush polymer of claim 1 having the formula: 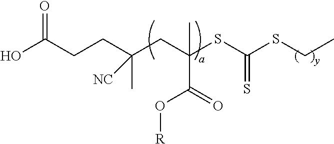
where R has the formula 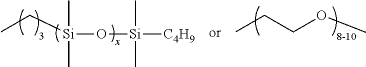
where x is an integer from about 5 to about 10; y is an integer from about 3 to about 11; and a is an integer from about 20 to about 300.

10. The bottlebrush polymer of claim 1 having the formula: 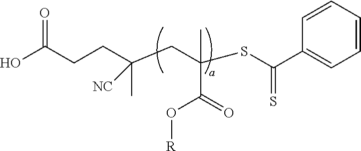
wherein R has the formula 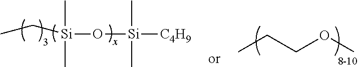
where x is an integer from about 5 to about 10; and a is an integer from about 20 to about 300.

11. The bottlebrush polymer of claim 1 having the formula: 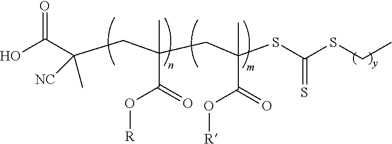
where R has the formula 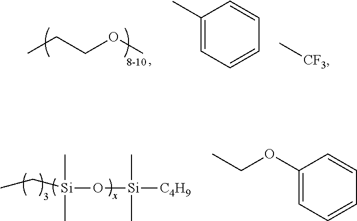
where x is an integer from 5 to 10; y is an integer from about 3 to about 11; n is a mole percent from about 70% to about 95%; and m is a mole percent from about 5% to about 30%.

12. The bottlebrush polymer of claim 1 having the formula: 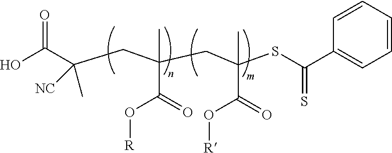
wherein R has the formula 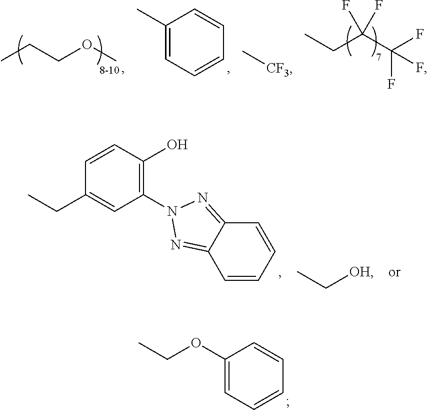
R′ has the formula 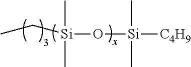
where x is an integer from about 5 to about 10; n is a mole percent from about 5% to about 30%; and m is an mole percent from about 70% to about 95% and n+m=100.

13. The bottlebrush polymer of claim 1 having the formula: 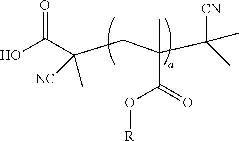
where R has the formula 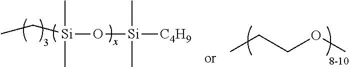
where y is an integer from about 5 to about 10; and a is an integer from about 20 to about 300.

14. The bottlebrush polymer of claim 2 having the formula 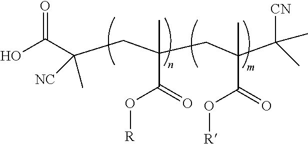
where R has the formula 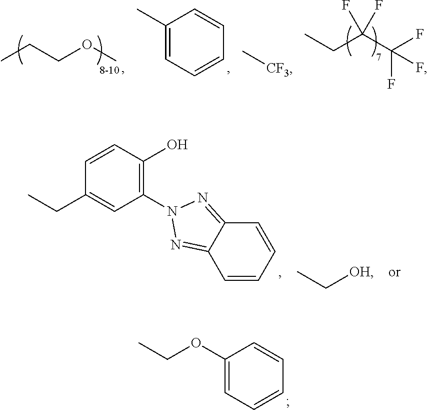
R′ has the formula 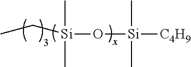
x is an integer from about 5 to about 10; n is a mole percent from about 70% to about 95%; and m is an mole percent from about 5% to about 30%.

15. The bottlebrush polymer of claim 2 having the formula: 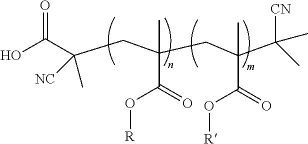
where R has the formula 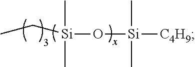
R′ has the formula 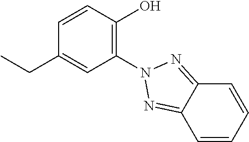 [SMILES: CCc1ccc(O)c(-n2nc3ccccc3n2)c1]
x is an integer from about 5 to about 10; n is a mole percent from about 70% to about 95%; and m is a mole percent from about 5% to about 30%.

16. The bottlebrush polymer of any one of claims 1 wherein said bottlebrush polymer is optically clear.

17. A filling material for use in an artificial lens comprising one or more optically clear bottlebrush polymers having a refractive index of from about 1.39 to about 1.48, and a complex viscosity of from about 0.5 to about 50 Pa.s.

18. The filling material of claim 17 wherein said artificial lens is an accommodating intraocular lens (A-IOL) or a presbyopia-correcting IOL.

19. The filling material of claim 17 wherein said one or more optically clear bottlebrush polymer comprises a homopolymer of a methacrylate macromolecule monomer selected from the group consisting of monomethacryloxypropyl terminated polydimethylsiloxane, asymmetric (PDMS-MA) and oligo(ethylene glycol) methacrylate (OEGMA) or a copolymer of at least one of PDMS-MA and OEGMA and at least one methacrylate or acrylate monomer selected from the group consisting of 2,2,2-trifluoroethyl methacrylate (TFEMA), 3,3,4,4,5,5,6,6,7,7,8,8,9,9,10,10,10-heptadecafluorodecyl methacrylate (HDFDMA), benzyl methacrylate (BzMA), 2-[3-(2H-Benzotriazol-2-yl)-4-hydroxyphenyl]ethyl methacrylate (BzTAzMA), ethyleneglycol phenylether methacrylate (EGPhEMA), hydroxyethyl methacrylate (HEMA), 2,2,2-trifluoroethyl acrylate (TFEA), 3,3,4,4,5,5,6,6,7,7,8,8,9,9,10,10,10-heptadecafluorodecyl acrylate (HDFDA), benzyl acrylate (BzA), 2-[3-(2H-Benzotriazol-2-yl)-4-hydroxyphenyl]ethyl acrylate (BzTAZA), ethyleneglycol phenylether acrylate (EGPhEA), hydroxyethyl acrylate (HEA), and combinations thereof, and having one or more end groups derived from a reversible addition fragmentation chain-transfer (RAFT) agent, wherein said bottlebrush polymer has a complex viscosity of from about 0.5 to about 30 Pa·s at 37° C. and is optically clear.

20. The filling material of claim 17 wherein said one or more optically clear bottlebrush polymer is a homopolymer or copolymer of a methacrylate macromolecule monomer selected from the group consisting of monomethacryloxypropyl terminated polydimethylsiloxane, asymmetric (PDMS-MA) and oligo(ethylene glycol) methacrylate (OEGMA) or a copolymer of at least one of PDMS-MA and OEGMA and at least one methacrylate or acrylate monomer selected from the group consisting of 2,2,2-trifluoroethyl methacrylate (TFEMA), 3,3,4,4,5,5,6,6,7,7,8,8,9,9,10,10,10-heptadecafluorodecyl methacrylate (HDFDMA), benzyl methacrylate (BzMA), 2-[3-(2H-Benzotriazol-2-yl)-4-hydroxyphenyl]ethyl methacrylate (BzTAzMA), ethyleneglycol phenylether methacrylate (EGPhEMA), hydroxyethyl methacrylate (HEMA), 2,2,2-trifluoroethyl acrylate (TFEA), 3,3,4,4,5,5,6,6,7,7,8,8,9,9,10,10,10-heptadecafluorodecyl acrylate (HDFDA), benzyl acrylate (BzA), 2-[3-(2H-Benzotriazol-2-yl)-4-hydroxyphenyl]ethyl acrylate (BzTAzA), ethyleneglycol phenylether acrylate (EGPhEA), hydroxyethyl acrylate (HEA), and combinations thereof, and having one or more end groups derived from a reversible addition fragmentation chain-transfer (RAFT) agent.

21. The filling material of claim 20 wherein said optically clear bottlebrush polymer is a copolymer of monomethacryloxypropyl terminated polydimethylsiloxane, asymmetric (PDMS-MA) and oligo(ethylene glycol) methacrylate (OEGMA) comprising from about 10 to about 95 mole percent PDMS-MA.

22. The filling material of claim 17 wherein said optically clear bottlebrush polymer has a complex viscosity of from about 0.5 to about 30 Pa·s at 37° C.

23. (canceled)

24. (canceled)

25. (canceled)

26. The filling material of claim 17 wherein said optically clear bottlebrush polymer has the formula: 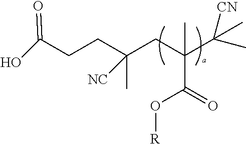
wherein R has the formula 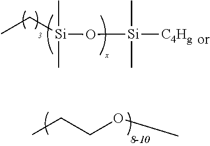
where x is an integer from 5 to 10, and a is an integer from about 20 to about 300.

27. The filling material of claim 17 wherein said optically clear bottlebrush polymer has the formula: 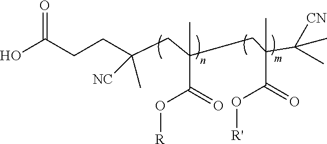
where R has the formula 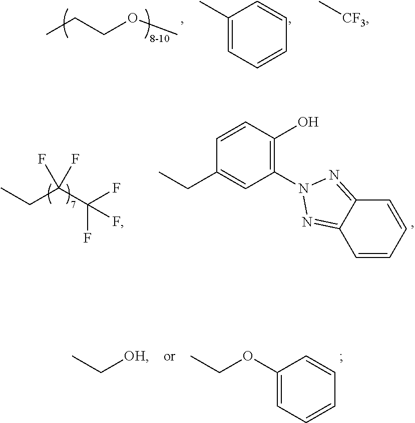
R′ has the formula 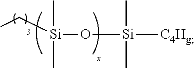
n is an mole percent from about 5% to about 30%; m is a mole percent from about 70% to about 95%; and y is an integer from about 5 to about 10.

28. An intraocular lens comprising: a filling medium and a capsular interface configured and dimensioned to be received within the natural eye capsule, and to be filled with the filling medium either prior to insertion in the eye or in situ, wherein said filling material comprises one or more optically clear bottlebrush polymers having a refractive index from about 1.39 to about 1.48 and a complex viscosity from about 0.5 Pa.s to about 50 Pa.s, wherein the capsular interface filled with the filling medium defines a predetermined optical power.

29. (canceled)

30. (canceled)

31. (canceled)

32. (canceled)

33. (canceled)

34. The intraocular lens of claim 28 which corrects for corneal astigmatism through different powers built along different meridians of the polymeric capsule interface or through filling medium with different refractive indexes in different compartments within the intraocular lens.

35. The intraocular lens of claim 28 wherein the filling medium comprises the residue of an ultra-violet (UV) light blocking methacrylate monomer and has the formula: 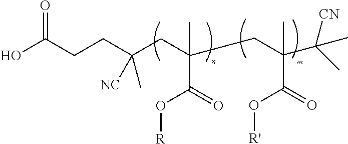
where R has the formula 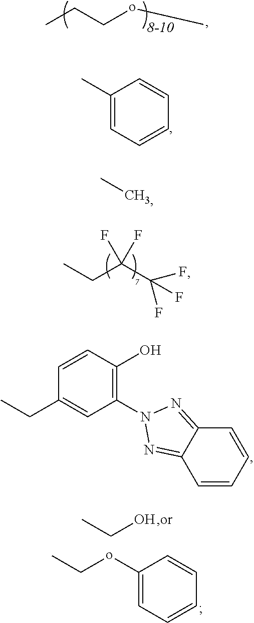
R′ has the formula 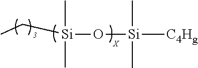
x is an integer from about 5 to about 10; n is a mole percent from about 70% to about 95%; and m is an mole percent from about 5% to about 30%.

36. The intraocular lens of claim 28 wherein said optically clear bottlebrush polymer is a homopolymer or copolymer of a methacrylate macromolecule monomer selected from the group consisting of monomethacryloxypropyl terminated polydimethylsiloxane, asymmetric (PDMS-MA) and oligo(ethylene glycol) methacrylate (OEGMA) or a copolymer of at least one of PDMS-MA and OEGMA and at least one methacrylate or acrylate monomer selected from the group consisting of 2,2,2-trifluoroethyl methacrylate (TFEMA), 3,3,4,4,5,5,6,6,7,7,8,8,9,9,10,10,10-heptadecafluorodecyl methacrylate (HDFDMA), benzyl methacrylate (BzMA), 2-[3-(2H-Benzotriazol-2-yl)-4-hydroxyphenyl]ethyl methacrylate (BzTAzMA), ethyleneglycol phenylether methacrylate (EGPhEMA), hydroxyethyl methacrylate (HEMA), 2,2,2-trifluoroethyl acrylate (TFEA), 3,3,4,4,5,5,6,6,7,7,8,8,9,9,10,10,10-heptadecafluorodecyl acrylate (HDFDA), benzyl acrylate (BzA), 2-[3-(2H-Benzotriazol-2-yl)-4-hydroxyphenyl]ethyl acrylate (BzTAZA), ethyleneglycol phenylether acrylate (EGPhEA), hydroxyethyl acrylate (HEA), and combinations thereof, and having one or more end groups derived from a reversible addition fragmentation chain-transfer (RAFT) agent.

37. (canceled)

38. The intraocular lens of claims 36 wherein said optically clear bottlebrush polymer comprises the residue of an ultra-violet (UV) light blocking methacrylate monomer.

39. (canceled)

40. The intraocular lens of claim 36 wherein said clear bottlebrush polymer has a complex viscosity from about 0.5 to about 15 Pa·s.

41. The intraocular lens of claim 36 wherein said optically clear bottlebrush polymer has a refractive index from about 1.43 to about 1.48.

42. The intraocular lens of claim 28 wherein said optically clear bottlebrush polymer has the formula: 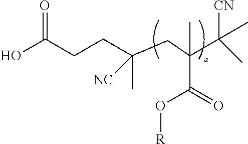
where R has the formula 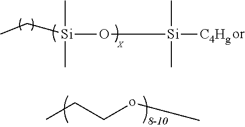
where x is an integer from about 5 to about 10; and a is an integer from about 20 to about 300.

43. The intraocular lens of claim 28 wherein said optically clear bottlebrush polymer has the formula: 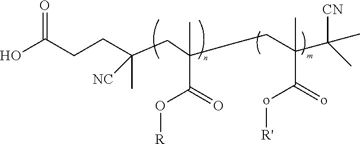
wherein R has the formula 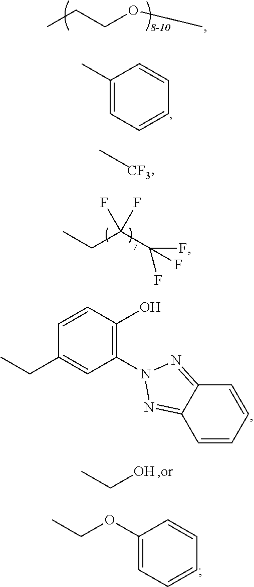
R′ has the formula 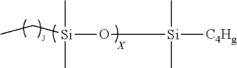
where x is an integer from 5 to 10; n is a mole percent from about 5% to about 30%; m is a mole percent from about 70% to about 95%.

44. The intraocular lens of claim 28 wherein said optically clear bottlebrush polymer has the formula: 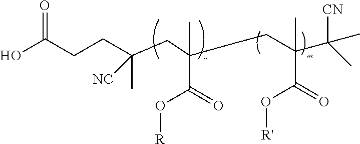
wherein R has the formula 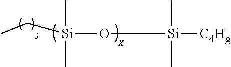
and R′ has the formula 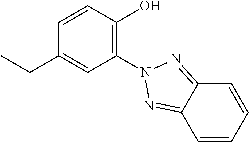
where x is an integer from 5 to 10; n is a mole percent from about 5% to about 30%; and m is a mole percent from about 70% to about 95%.
